(19) United States
(12) Patent Application Publication
(10) Pub. No.: US 2025/0177461 A1
(43) Pub. Date: Jun. 5, 2025

(54) PARTICLE COMPRISING A VIRUS

(71) Applicant: UNIVERSITY OF BATH, Bath (GB)

(72) Inventors: Asel SARTBAEVA, Bath (GB); Aswin DOEKHIE, Bath (GB); Matthew SLADE, Bath (GB); Stephen WELLS, Bath (GB)

(73) Assignee: UNIVERSITY OF BATH, Bath (GB)

(21) Appl. No.: 18/834,526

(22) PCT Filed: Jan. 24, 2023
(86) PCT No.: PCT/EP2023/051699

§ 371 Date: Jul. 30, 2024

(30) Foreign Application Priority Data
Jan. 31, 2022 (GB) 2201188.6

(51) Int. Cl. A61K 35/76; A61K 9/50; C12N 7/00

(52) U.S. Cl. CPC A61K 35/76; A61K 9/501; C12N 7/00; C12N 2795/18032

(57) ABSTRACT

A particle containing a virus encapsulated in an amorphous silica shell is described, wherein the amorphous silica shell is directly deposited about the surface of the virus. Also described is a method of producing a virus encapsulated in an amorphous silica shell, the method comprising enriching or purifying a virus; suspending the virus in buffer, hydrolysing a silica precursor directly contacting the hydrolysed silica precursor with the surface of the virus in buffer and encapsulating the virus in an amorphous silica shell.

### BACKGROUND

Virus-based therapeutics are increasingly being used in medicine. A first type of viruses used in medicine are bacteriophages; these can be used in “phage therapy” to treat bacterial infection. Many well-studied phages show activity against the most problematic bacteria including those that have multidrug or antibiotic resistance. One of the biggest differences between bacteriophage and chemical antibiotics is that bacteriophage have a narrow host range. Each phage species is only infective against a handful of bacterial strains. This characteristic can be extremely beneficial because it can lead to the successful treatment of the bacteria causing infection while allowing “good bacteria”, such as those that constitute the gut microbiome, to remain unharmed. This prevents many of the unpleasant side effects associated with chemical antibiotics.

Bacteriophages have different characteristics and morphologies, however, a large majority of phages (such as T4 or phage K) show a tailed morphology consisting of an icosahedral head that contains the genetic material, a tail, and a baseplate. Additionally, long tail fibres may extend from the baseplate acting as detection apparatus, searching for host organisms. A second known morphology is the spherical phage, such as MS2. This phage is structurally very similar to many mammalian viruses and has a spherical capsid to store genetic material.

Other viruses include mammalian viruses which can be used for vaccination to establish protective immunity. Live attenuated or whole virus vaccines contain a version of the living virus that has been weakened or deactivated so that it does not cause serious disease. Other vaccines make use of a mammalian virus vector which can deliver genetic instructions to the body's cells.

However, a drawback in using viruses as therapeutics is their thermal instability, since both the virus capsid and genetic material are prone to degradation upon exposure to heat. As a result, vaccines often need to be refrigerated for storage and transport. Certain viruses, including, bacteriophage MS2, are only viable for brief periods of time even at refrigeration temperatures, and as a result, deep-freezing in liquid nitrogen is required for long-term storage. This vastly increases the cost of storage and transport of these phages and prevents their use in settings where such facilities are unavailable.

### SUMMARY OF INVENTION

According to a first aspect of the invention, there is provided a particle comprising a virus encapsulated in an amorphous silica shell, wherein the amorphous silica shell is directly deposited about the surface of the virus.

According to a second aspect of the invention, there is provided a method comprising enriching or purifying a virus, and suspending the virus in buffer, hydrolysing a silica precursor directly contacting the hydrolysed silica precursor with the surface of the virus in buffer and encapsulating the virus in an amorphous silica shell.

The method of the second aspect can be used to produce the particles of the first aspect.

According to a third aspect of the invention, there is provided use of the method of the second aspect for preservation or storage of a virus.

According to a fourth aspect of the invention, there is provided a particle formed by the method of the second aspect.

According to a fifth aspect of the invention, there is provided a dry powder comprising the one or more particles of the first and/or fourth aspect.

According to the sixth aspect of the invention, there is provided a pharmaceutical composition comprising one or more particles according to the first and/or fourth aspect. In some embodiments, the pharmaceutical composition comprises at least two particles according to the first aspect and/or the fourth aspect, wherein the at least two particles comprise different viruses, for example, two different bacteriophages.

According to the seventh aspect of the invention, there is provided the particle of the first and/or fourth aspect, the dry powder of the fifth aspect or the pharmaceutical composition of the sixth aspect for use in therapy.

According to the eighth aspect of the invention, there is provided the particle of the first and/or fourth aspect, the dry powder of the fifth aspect or the pharmaceutical composition of the sixth aspect for use as an antibiotic medicine (i.e., an antibiotic medicament), wherein the virus is a bacteriophage.

According to the aspect of the invention, there is provided the non-therapeutic use of the particle of the first and/or fourth aspect, the dry powder of the fifth aspect or the pharmaceutical composition of the sixth aspect for use as an antimicrobial.

Also disclosed herein is a network of the particles of the first and/or fourth aspect. In some embodiments, the network of particles is in the form of a chain.

Also disclosed herein is a virus stabilized by an amorphous silica shell. The virus and amorphous silica shell may be as defined herein.

Also disclosed herein is a particle comprising a bacteriophage encapsulated in an amorphous silica shell, wherein the amorphous silica shell is directly deposited about the surface of the bacteriophage.

Also disclosed herein, is provided a particle comprising a virus encapsulated in an amorphous silica shell, wherein the particle is free of a polycationic polymer (e.g., lysine).

### Advantages of One or More of the Above Aspects

The present invention provides a particle comprising a virus that is substantially stabilized in an amorphous silica shell as compared with a native, unencapsulated virus, with improved thermal stability. Surprisingly, after silica encapsulation and release, the virus is still viable and capable of replication.

The present invention also provides a method of producing such particles. The method used to produce the particles is a modified sol-gel method. In this method, hydrolysed silica precursors (i.e., silica monomers) first directly interact and are directly deposited about the surface on the virus. The silica shell then grows or polymerises about the surface of the virus. The end result is the formation of a rigid amorphous silica shell about the virus, which protects the virus from surrounding denaturing conditions and prevents deterioration of the viral structure and degradation of the genetic material.

The method and the resultant particle therefore differs from typical methods used for silica encapsulation which involve an intermediate positively charged template which surrounds the virus particle prior to silica deposition. The method and resultant particle also differ from methods in which a silica structure is pre-formed and subsequently loaded with a biological payload. Further, in order for silica encapsulation of viruses to be achieved, and different to previous silica encapsulation methods, an enriching or purifying step was found to be key for successful silica encapsulation and particle growth. Other parameters were also found to be particularly preferred or optimal for the silica encapsulation of viruses such that they can be subsequently released with a high recovery rate.

This is the first time that such a silica encapsulation/ensilication process has been demonstrated for such a large complex biological structure that contains genetic material. Earlier studies and investigations have only demonstrated ensilication for smaller and simple protein-based biological targets which lack genetic material. Importantly and unexpectedly, the virus was able to withstand the encapsulation and enrichment process and were able to replicate after release. This therefore provides evidence that the genetic material of the virus is able to remain intact and survive the silica encapsulation process. Even more surprisingly, the silica encapsulated viruses disclosed herein were able to produce plaques after release even when stored at ambient temperatures for long periods of time, or when subjected to high temperatures. The particles described herein therefore provide a method of preserving and stabilizing viruses, eliminating the need for refrigeration or cold-chain for storage or transportation.

Surprisingly, the particles of the present invention, i.e., formed by the present method, substantially differ in properties from previous ensilicated/silica encapsulated particles comprising a protein or polypeptide. The particles of the present invention were found to have a larger median particle size, with the silica shell also being porous in nature (i.e., and therefore substantially more porous in nature than for silica encapsulated proteins or polypeptides). For example, the particles disclosed herein may have a median particle size of at least 250 nm as determined by field-emission scanning electron microscope (SE-FEM), more particularly wherein the median diameter is from 250 nm to 1000 nm, or from 250 to 600 nm, or from 300 nm to 500 nm, or about 400 nm.

One or more embodiments of the present invention may also have one or more of the following features or additional advantages.
In the particles disclosed herein, the virus encapsulated in the silica shell can be protected from surrounding conditions, such as temperature or pH. In particular, the virus encapsulated in the silica shell is thermally stable and has improved thermal stability as compared with the native unencapsulated virus. In some examples, viruses are shown to be stable after heating at 90° C. for 30 minutes and/or long-term storage at ambient temperature (e.g., 21° C.). As a result, the particles disclosed herein can be used to preserve and/or stabilize viruses, eliminating the need for cold-chain during storage or transport.
The particles disclosed herein may comprise a virus which is a bacteriophage. This is useful as bacteriophages can be used in therapy, for example, to treat bacterial infection.
The particles disclosed herein may be free of a polycationic polymer, more particularly polylysine. This is advantageous because the addition of a polycationic polymer increases the complexity of the particle and/or formulation and increases the likelihood of side-effects in therapy while making regulatory approval more difficult to obtain. This also provides a particle that has lower cost and avoids any potential undesirable interaction between the polymer and the silica which might interfere with the formulation of the particle and/or the release of the payload.
The particles and methods disclosed herein are applicable to different types of viruses. Indeed, as is demonstrated in the Examples, the virus may be a DNA virus or an RNA virus and may have various shapes or morphologies. In some examples disclosed herein, the virus is a highly complex head and tail virus (e.g., of the myoviridae family, such as bacteriophage K), whereas in other examples disclosed herein, the virus is a small spherical virus (e.g., of the fiersviridae family, such as MS2). This indicates that the method disclosed herein is broadly applicable to different types of viruses. The methods disclosed herein are also broadly applicable to mammalian viruses, since these closely resemble MS2 in structure. The particles disclosed herein may also comprise viruses of different molecular weights, including viruses with smaller molecular weights (e.g., MS2 with a molecular weight of ˜50 kDa) and larger molecular weights (e.g. phage K) with a molecular weight greater than 250 kDa or greater than 500 kDa (e.g. phage K)
In the method disclosed herein, the virus in the buffer is preferably at a concentration greater than 1×10⁷ PFU/ml, more preferably greater than 1×10⁸ PFU/ml, or more preferably greater than 1×10⁹ PFU/ml, or more preferably at a concentration from 1×10⁹−1×10¹⁰ PFU/mL. A higher concentration of virus in buffer is found to contribute to a higher recovery rate of the virus, once released.
The method disclosed herein may include a buffer comprising one or more salts, preferably one or more dicationic or monocationic metal salts. The one or more salts may stabilize the virus, increase the recovery rate of the virus, increase the titre of propagated solutions, and/or promote particle growth during silica encapsulation. In particular, the method disclosed herein may include a buffer that comprises one or more of a magnesium salt, a calcium salt or a sodium salt. Dicationic salts comprising magnesium or calcium are believed to contribute to the stability of the lysate and the absence of these salts reduces the titre of the propagated phage solutions. The presence of certain salts (e.g., sodium ions) is also believed to i) further contribute to the stability of liquid lysates and ii) promote particle growth, where the salt ions can act as bridges between silica particles. In some examples disclosed herein, the buffer is an SM buffer or a modified SM buffer (e.g., SM buffer where 50 mM Trizma is substituted with glycine). Other suitable buffers may include PBS buffer, imidazole buffer (e.g., 50 mM imidazole buffer), bis-tris buffer or a sucrose buffer. Such buffers are found to be more effective for silica encapsulation of viruses as compared with a Tris only buffer
In the method disclosed herein, the ratio of hydrolysed silica to virus in buffer may be from about 1:1 to 1:250, or from 1:75 to 1:150, for example, about 1:100. For higher ratios of virus in buffer to silica, the ensilication process is believed to be less uniform.
In the method disclosed herein, the pH of the contacting step is preferably slightly alkaline, for example, wherein the pH is greater than 7, more preferably between 7.25 and 8.5, or between 7.25 and 8, for example, about 7.5. pH is found to influence the rate of particle growth and can improve the stability of the phage. While silica deposition and polymerization still works below pH 7 (e.g., at a pH between 6 and 7), this process is much slower. In contrast, at higher pH's (e.g., at a pH above 9), the integrity of the virus is affected.
In some embodiments of the method disclosed herein, the enriching or purifying step is a polyethylene glycol (PEG)-based enrichment, e.g., using PEG 6000. This enrichment method is very convenient and quick, using easily obtainable equipment and reagents with very little active man hours required. This method allows for an easy buffer exchange between the growth solution (TSB) and the working buffer (e.g., SM). In alternative embodiments, the enriching or purifying step is by chloroform purification, filtration methods, size exclusion chromatography, ion-exchange chromatography or gradient centrifugation, which are other methods that can be used to produce a product with higher purity.
In the method disclosed herein, the enriching or purifying step is such that the virus in buffer has a polydispersity index (PDI) of less than 0.2, more preferably less than 1.5. A lower PDI is believed to improve the silica encapsulation process as the silica is directed towards the target as opposed to any debris or contaminants that may be present.
In the method disclosed herein, the silica precursor is preferably a tetra-alkyl orthosilicate, more preferably tetra-ethyl orthosilicate, as TEOS produces a less toxic and hazardous byproduct of ethanol as opposed to TMOS where the byproduct is methanol.
In the method disclosed herein, the contacting step is preferably carried out for at least 5 minutes, or at least or equal to 10 minutes. The contacting step may be carried out in less than 30 minutes, or less than 20 minutes. As a result, the formation of particles is very convenient and quick.
In the method disclosed herein, the method is free of a templating step, for example, using a polycationic polymer such as polylysine. This is in contrast to known silica encapsulation methods where the polylysine is first used to form an intermediate layer around the biological molecule or entity before silica deposition. The resulting method of the present invention does not involve a templating step and is therefore simpler and less complex.
The particles herein may be in the form of a dry powder comprising said particles. The dry powder may further improve the long-term storage of the particles. The dry powders can also easily be formed since the particles of the present invention precipitate in solution, followed by vacuum filtration and drying.
The particles herein may be formulated in a pharmaceutical composition. The pharmaceutical composition may comprise one or more pharmaceutical carriers or adjuvants to aid delivery of the particles.
The pharmaceutical composition disclosed herein may comprise one or more particles as disclosed herein. Advantageously, the pharmaceutical composition can comprise two particles as disclosed herein, wherein the at least two particles comprise different viruses. This allows a cocktail of different viruses to be stabilized or administered at the same time (i.e., for an antibacterial action against different types of bacteria). In the case of bacteriophage this is particularly beneficial to target a wider range or type of bacteria.

### BRIEF DESCRIPTION OF FIGURES

FIG. 1 shows an example method in accordance with the present invention, to form example particles of the present invention. A solution of prehydrolysed TEOS is added to a bacteriophage K suspension and mixed gently for 10 minutes. During this time silica surrounds the phage molecules and forms silica particles that begin to precipitate out of solution. After 10 minutes the solution is vacuum filtered and air dried.

FIG. 2 shows Top: a schematic of a head and tail phage (e.g., T4 or Phage K with features 1—protein capsid (i.e., capsid head), 2—genetic material, 3—collar, 4—whiskers, 5—tail outer, 6—baseplate, and 7=legs, and Bottom: a spherical phage (MS2) with 1b—protein capsid, 2b genetic material, and 8—maturation protein.

FIG. 3 shows the DLS results of size analysis of 0.45 μm filtered enriched bacteriophage K sample used in silica encapsulation/ensilication. Left) shows the correlogram of the sample analysis, and Right) depicts the size analysis of the sample. The bottom trace shows the estimated size as measured by scattered light intensity and the top trace the size as measured by particle volume.

FIG. 4 shows a transmission electron microscope (TEM) image of propagated and enriched bacteriophage K.

FIG. 5 shows a Field-Emission Scanning Electron Microscope (FE-SEM) image of fully silica encapsulated/ensilicated and dried bacteriophage K with magnification at 30,000×.

FIG. 6 shows a graph showing the effect of pH on the speed of particle size growth during silica encapsulation/ensilication of bacteriophage K, with analysis taken using dynamic light scattering (DLS) instrumentation. Each point is an average of an 11 second measurement window. The measured silica encapsulations/ensilications used enriched phage K lysates suspended in SM buffer and a 1:100 ratio of silica to phage solutions. The rate of silica polymerisation and deposition onto the phages increases with concentration of [OH—] until the particles become mutually repulsive, seen at approximately pH 9.

FIG. 7 shows an FE-SEM image showing silica encapsulated/ensilicated MS2 particles, with a magnification of 15,000×.

FIG. 8 demonstrates Left) a plaque assay of native bacteriophage K in varying dilutions, wherein the top row shows full confluence of bacterial lysis by the phage; the middle row shows semi confluence of lysis, and the bottom row shows ideal counting conditions, with individual plaques visible; Right) a plaque assay of a silica encapsulated/ensilicated sample after release (i.e., Sample No. 2 of Table 1)

FIG. 9 shows plaque assay results obtained from: Left) silica encapsulated/ensilicated, heated, and released bacteriophage K (i.e., Sample No. 8 of Table 1), in dilutions ranging from neat to ×10⁻²; and Right: plaque assay results of heated native (i.e., unensilicated) phage k lysates.

FIG. 10 shows an image of agar plates used in the plaque assay of ensilicated, released, and propagated MS2 using varying concentrations of HF in their release. a) Shows the results from a sample taken of 5 hours after propagation. b) Shows the results of a sample taken after 24 hours of propagation.

FIG. 11 shows images of plaque assay agar plates of a) Ensilicated, heated at 90° C. for 30 minutes, and released bacteriophage K; b) Heated at 90° C. for 30 minutes native phage K lysates; c) Ensilicated released bacteriophage K with clearly visible plaques and d) Native non-heated phage K lysates.

FIG. 12 shows Top) a graph showing a plot of dV(log d) against pore diameter for the BJH isotherm of bacteriophage K ensilicated using a PHOS:lysate ratio of 1:10. d is pore diameter, and V is pore volume, and Bottom) a graph showing a plot of dV(log d) against pore diameter for the BJH isotherm of phage K ensilicated using a PHOS:lysate ratio of 1:40. d is pore diameter, and V is pore volume

FIG. 13 shows the growth of key measurement points of phage virions over time of the silica encapsulation/ensilication process. Tail width is shown by the bottom line with measurements on the primary axis on the left-hand side and head diameter (top to bottom) is shown by the top line with measurements on the axis on the right-hand side. Error bars are 2×standard deviation of measurements.

FIG. 14 shows transmission electron microscope (TEM) images captured of bacteriophage K after 1 minute of silica encapsulation/ensilication. Values are in nm. H=head, T=tail and S=silica nodule

FIG. 15 shows TEM images of bacteriophage K after 2 minutes of silica encapsulation/ensilication. Values are in nm. H=head, T=tail and S=silica nodule

FIG. 16 shows TEM images captured of bacteriophage K after 5 minutes of silica encapsulation/ensilication. Values are in nm. H=head, T=tail and S=silica nodule

FIG. 17 shows TEM images of bacteriophage K after 7 minutes of silica encapsulation/ensilication. Values are in nm. H=head, T=tail and S=silica nodule

FIG. 18 shows an ITEM image of phage K after 7 minutes of ensilication. The outline shows 2 distinct individual phage virions being incorporated into the same silica cluster network.

### DETAILED DESCRIPTION

As defined herein the term “ensilication” can be used interchangeably with “silica encapsulation”.

As defined herein “Bacteriophage K” is a double stranded DNA based bacteriophage of the myoviridae family. Bacteriophage K exhibits a classic tailed phage super structure in its morphology, having an icosahedral head, long contractile tail, and hexagonal/star shaped base plate, measuring ˜300 nm from tail to head, resembling bacteriophage T4. Phage K is polyvalent, able to infect a range of host strains, and infective to Staphylococcus aureus. Bacteriophage K is otherwise known or referred to as Staphylococcus aureus phage K, Staphylococcus virus K or Phage K.

As defined herein “Bacteriophage MS2” is an icosahedral/isometric single-stranded RNA based bacteriophage of the fiersviridae family. Bacteriophage MS2 is otherwise known or referred to as MS2.

As defined herein “native virus” refers to a virus that has not been silica encapsulated/ensilicated.

The terms “treatment” and “treating” herein refer to an approach for obtaining beneficial or desired results in a subject, which includes a prophylactic benefit and optionally also a therapeutic benefit.

“Prophylactic benefit” refers to delaying or eliminating the appearance of a disease or condition, delaying or eliminating the onset of symptoms of a disease or condition, slowing, halting, or reversing the progression of a disease or condition, or any combination thereof.

“Therapeutic benefit” refers to eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the patient may still be afflicted with the underlying disorder.

The term “comprising” (and related terms such as “comprise” or “comprises” or “having” or “including”) includes those embodiments, for example, an embodiment of any composition of matter, composition, method, or process, or the like, that “consists of” or “consists essentially of” the described features. The term “comprises” or “comprising” can be used interchangeably with “includes”.

When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale.

The features of any dependent claim and/or embodiments described herein are intended to be readily combined with the features of any of the independent claims or other dependent claims described herein unless context clearly indicates otherwise.

Unless defined otherwise, all technical and scientific terms used herein have the same meanings as commonly understood by one of ordinary skill in the art to which this invention belongs. Abbreviations used herein have their conventional meaning within the chemical and biological arts, unless otherwise indicated.

### Particle

Disclosed herein is a particle comprising a virus encapsulated in an amorphous silica shell. The particle may be formed using the methods as disclosed herein.

The amorphous silica shell can be directly deposited about the surface of the virus. As defined herein the term “directly deposited” refers to the silica being deposited on the surface on the virus, for example, the protein capsid of the virus, as opposed to being deposited on an intermediate polymer layer between the virus and the silica shell. For the particles described herein, the amorphous silica shell electrostatically interacts with at least one positively charged amino acid on the virus surface.

The particles described herein may comprise a single virus or a plurality of viruses. In some embodiments, the particle comprises a single virus. In some embodiments, the particle comprises two or more, or three or more, or four or more, or five or more viruses.

In some examples, the particle of the present invention may have a larger median particle size than previously reported silica encapsulated biomolecules. In some embodiments, the particle has a median particle size of at least 250 nm as determined by field-emission scanning electron microscope (SE-FEM), or of at least 300 nm, or at least 350 nm, or at least or equal to 400 nm as determined by field-emission scanning electron microscope (SE-FEM). In some embodiments, the particle has a median particle size of between 250 nm and 1000 nm as determined by field-emission scanning electron microscope (SE-FEM), or from 250 nm to 750 nm, or from 275 nm to 600 nm, or from 300 nm to 500 nm, or in some examples, about 400 nm as determined by field-emission scanning electron microscope (SE-FEM). In some embodiments, the particle has a median particle size of less than 1000 nm as determined by field-emission scanning electron microscope (SE-FEM), or less than 900 nm, or less than 800 nm, or less than 700 nm, or less than 600 nm, or less than 500 nm as determined by field-emission scanning electron microscope (SE-FEM).

In some examples, the particles of the present invention may have increased porosity as compared to previously reported silica encapsulated biomolecules. In some embodiments, the amorphous silica shell is porous, (i.e., comprising one or more pores). In some embodiments, the silica shell is permeable to water. In some embodiments, the silica shell is permeable to small molecules, i.e., molecules with a molecular weight less than 500 Da, or less than 300 Da, or less than 100 Da, or permeable to molecules with a molecular weight of less than 50 Da. In some embodiments, the silica shell has a median pore size of less than 20 nm, or less than 15 nm, or less than 10 nm as determined using a Barrett-Joyner-Halina isotherm. In some embodiments, the particles disclosed herein may have a surface area of greater than 25 m²/g, or greater than 30 m²/g, i.e., as determined by BET. In some embodiments, the particles disclosed herein may have a surface area of between 25 m²/g and 40 m²/g, or from 30 m²/g to 40 m²/g.

### Virus

The particle disclosed herein comprises a virus. As defined herein the term “virus” can be used interchangeable with a virion. A virus is a biological structure that comprises at least genetic material (i.e., nucleic acid) and a capsid (i.e., a protein coat) which surrounds the genetic material. The virus may further comprise an outside envelope of lipids. In some embodiments, the virus (e.g., a myoviridae virus) may further comprise a collar, tail, baseplate or a combination thereof. In some embodiments, the virus may further comprise fibres (e.g., whiskers or legs). The term “virus” is different and distinguished from a “virus-like particle” because a virus additionally comprises genetic material. For the avoidance of doubt, the term “virus” does not include a virus-like particle.

The virus may be any suitable virus. The virus may be a filamentous virus, an isometric (i.e., icosahedral) virus, a rod-shaped virus, a bottle-shaped virus, a lemon-shaped virus, a pleomorphic virus, or a head and tail virus. In some examples, the virus is a head and tail virus, or an isometric virus.

In some embodiments, the virus is a non-enveloped virus. In some embodiments, the virus is a non-enveloped isometric virus. (e.g., MS2).

In other embodiments, the virus is an enveloped virus. In some embodiments, the virus is a non-enveloped head and tail virus (e.g., phage K) In some embodiments, the virus is a bacteriophage (i.e., a virus that is capable of infecting and replicating within bacteria and archaea).

In some embodiments, the virus (i.e., bacteriophage) is of the order belfryvirales, caudovirales, halopanivirales, haloruvirales, kalamavirales, ligamenvirales, mindivirales, norzivirales, petitvirales, primaviriales, timlovirales, tubulavirales, vinavirales or durnavirales. In some examples, the bacteriophage is of the order norzivirales (e.g., MS2) or caudovirales (e.g., phage K).

In some embodiments, the virus (i.e., bacteriophage) is of the family selected from turriviridae, ackermannviridae, autographviridae, chaseviridae, demerecviridae, drexlerviridae, guenliviridae, herelleviridae, myoviridae, siphoviridae, podoviridae, rountreeviridae, salasmaraviridae, schitoviridae, zobellviridae, sphaerolipoviridae, simuloviridae, matshushitaviridae, pleolipoviridae, tectiviridae, lipothrixviridae, rudiviridae, cystoviridae, atkinsviridae, duinviridae, fiersviridae, solspiviridae, microviridae, tristomaviridae, blumeviridae, steitzviridae, inoviridae, paulinoviridae, plectroviridae, corticoviridae, ampullaviridae, autolykiviridae, bicaudaviridae, clavaviridae, finnlakeviridae, fuselloviridae, globuloviridae, guttaviridae, halspviridae, plasmaviridae, portogloboviridae, thaspiviridae, spiraviridae. In some examples, the virus is of the family fiersviridae (e.g., MS2) or myoviridae (e.g., phage K) In some embodiments, the virus (i.e., bacteriophage) is of the species selected from bacteriophage MS2, bacteriophage QP (i.e., Qbeta), bacteriopage T4, bacteriophage K, bacteriophage f2, bacteriophage R17 and bacteriophage GA. In other embodiments, the bacteriophage species may be selected from bacteriophage mu, P1 phage, bacteriophage P2, enterobacteria A, bacteriophage T5, bacteriophage HK96, bacteriophage N15, bacteriophage T7, bacteriophage T3, bacteriophage D29 or bacteriophage P22 In some examples, the virus is bacteriophage MS2 or phage K.

In some embodiments, the virus is a mammalian virus. In some embodiments, the virus may be of the family selected from flaviviridae, coronaviridae (i.e., coronavirus), adenoviridae, herpesviridae, poxviridae, parvoviridae, reoviridae (i.e., including rotavirus), retroviridae, togaviridae, orthomyxoviridae (i.e., including the influenza virus), hepadnaviridae. In some embodiments, the mammalian virus is an isometric mammalian virus.

In some embodiments, the mammalian virus is a vaccine or part of a vaccine (i.e., or the mammalian virus is suitable for use as a vaccine to establish immunity in humans/mammals). In some embodiments, the mammalian virus is an inactivated/dead virus. In other embodiments, the mammalian virus is a live virus, preferably a live attenuated virus. In some embodiments, the mammalian virus is a viral vector vaccine.

The virus described herein contains genetic material. In some embodiments, the virus is an RNA virus (i.e., comprising RNA genetic material). The RNA virus may comprise single-stranded or double-stranded RNA. In some examples, the RNA virus comprises single-stranded RNA, which may be positive sense RNA or negative sense RNA. In some examples, the RNA virus comprises positive-sense single stranded RNA (for example, phage MS2). The RNA virus may comprise linear or circular RNA, e.g., linear dsRNA, linear ssRNA, circular dsRNA or circular ssRNA. In some embodiments, the virus is a DNA virus (i.e., comprising DNA genetic material). The DNA virus may comprise single-stranded DNA (ssDNA) or double-stranded DNA (dsDNA). The DNA virus may comprise linear or circular DNA, e.g., linear dsDNA, linear ssDNA, circular dsDNA or circular ssDNA. In some examples, the DNA virus comprises double-stranded linear DNA (e.g., bacteriophage K).

The virus may have any suitable molecular weight. In some embodiments, the virus has a molecular weight greater than 55 kDa, or greater than 100 kDa, or greater than 250 kDa, or greater than 500 kDa, or greater than 1 MDa, or greater than 25 MDa, or greater than 50 MDa, or greater than 100 MDa. In some embodiments, the virus has a molecular weight less than 250 MDa, or less than 200 MDa, or less than 100 MDa, or less than 50 MDa, or less than 25 MDa, or less than 1 MDa, or less than 500 kDa, or less than 250 kDa, or less than 100 kDa. In some embodiments, the virus has a molecular weight from 55 kDa to 250 MDa. In some examples, the virus has a molecular weight from 1 MDa to 250 MDa, or from 10 MDa to 250 MDa. In some examples, the virus has a molecular weight of approximately about 33 MDa (i.e. phage K) or 56 kDa (i.e., MS2).

In preferred embodiments, the virus is viable and/or shows no or minimised loss in biological activity, (i.e., after release from the silica shell). In preferred embodiments, the virus is capable of infecting a host cell (i.e., after release of the virus from the silica shell). In preferred embodiments, the virus is capable of replication in a host cell (i.e., after release of the virus from the silica shell). For embodiments where the virus is a bacteriophage, the host cell is a bacteria or archaea. In some embodiments, the bacteria is E. coli (e.g., male E. coli), Staphylococci (e.g. S. Aureus), Streptococci, Pseudomonas, Shigella or Vibrio.

For embodiments in which the virus is a mammalian virus, the host cell is a mammalian cell. In some embodiments, the mammalian cell is a human cell. In some embodiments, the mammalian cell is a cow cell, sheep cell, pig cell, goat cell, dog cell, cat cell, donkey cell, deer cell, horse cell or rodent cell (e.g., mouse cell or guinea pig cell), fox cell, wolf cell, bat cell, coyote cell, raccoon cell, skunk cell, ferret cell, monkey cell, primate cell, hare cell, rabbit cell, bear cell, mongoose cell.

### Silica Shell

In some embodiments, the silica shell is directly deposited about the entire surface of the virus. In alternative embodiments, the silica shell is directly deposited about a major portion of the surface of the virus, i.e., about 50% of the surface of the virus, or about 60%, or at about 70%, or about 80%, or about 85%, or about 90%, or about 90%, or about 99%, or about 99% of the surface of the virus. This may be determined by TEM-EDX (transmission electron microscopy energy-dispersive X-ray spectroscopy) or by FE-SEM-EDX (field emission scanning electron microscopy energy-dispersive X-ray spectroscopy).

The silica shell is an amorphous silica shell. An amorphous silica shell refers to a silica shell that is non-crystalline. The silica shell is preferably a polymerised matrix of silica, i.e., covalently bonded silica. In the present invention, the silica shell is in direct contact with the surface of the virus, for example, the protein capsid of the virus, and more specifically at least one positive amino acid residues which make up the protein capsid of the virus. The direct contact is preferably an electrostatic interaction. In preferred embodiments, the silica shell is not covalently bonded to the surface of the virus.

In some embodiments, the silica shell is non-uniform or substantially non-uniform (i.e., the silica shell has an unequal thickness about the surface of the virus). For example, for head and tail viruses, the silica shell may be thicker about the head as compared with the tail. In embodiments where the virus comprises a baseplate, the silica shell may also be thicker about the baseplate as compared to other parts of the virus surface (e.g., the tail).

The silica shell may have any suitable morphology. In the examples disclosed herein, the silica shell has a spheroid morphology. In some embodiments, the silica shell has a spherical morphology. In some embodiments, the silica shell has an ellipsoid morphology.

### Components of the Particle

As defined above, the particle comprises a virus and an amorphous silica shell.

In some embodiments, particle comprises one or more salts. In preferred embodiments, the one or more salts may comprise a dicationic or monocationic metal ion. In some embodiments, the dicationic metal ion is or comprises a magnesium ion or a calcium ion or a combination thereof. In some embodiments, the monocationic metal ion is or comprises a sodium ion. In some embodiments, the one or more salts comprises one or more of a magnesium ion, a calcium ion and a sodium ion. In some embodiments, the particle comprises the components of a buffer, for example, SM buffer or any buffer as otherwise described herein. In some examples, the one or more salts comprise magnesium sulfate and/or sodium chloride. The presence of certain salts is believed to contribute to the stability of liquid lysates and may also promote particle growth, where the salt ions can act as bridges between silica particles.

In some embodiments, the particle may further comprise one or more small molecules. In some embodiments, the one or more small molecules may be an amino acid. In some embodiments, the amino acid is glycine. In some embodiments, the electrostatic interaction may be facilitated by the presence of the small molecules such as an amino acid, or more specifically glycine. In some embodiments, the particle may be free of Trizma (i.e. tromethamine).

In some embodiments, the particle may further comprise a stabilizing component (i.e., to stabilize or further stabilize the virus). In some embodiments, the stabilizing component may comprise gelatin.

In preferred embodiments, the particle is free of a polycationic polymer material. In preferred embodiments, the particle is free of polylysine.

In some embodiments, the particle may comprise one or more excipients. The one or more excipients may be selected from emulsifiers, osmolytes, co-solvents, antimicrobials or a combination thereof. Examples of emulsifiers may be Tween 20 or Tween 80. Examples of osmolytes include sucrose. Examples of co-solvents may be sorbitol and/or glycerol. Examples of antimicrobials include phenol and/or benzethonium chloride). In some embodiments, the particle may comprise one or more adjuvants. In some embodiments, the adjuvants may be selected from alum, a monophosphoryl lipid (e.g., MPLA), a squalene based-adjuvant (e.g., AS03 or MF59), AS04 (e.g., a combination of MPLA and alum), or CpG DNA adjuvant (e.g., CpG 1018).

### Properties

In the particle disclosed herein, the virus may be protected from surrounding conditions. In some examples, the virus may be protected from temperature. In some examples, the virus may be protected from an acidic or basic pH.

In the particle disclosed herein, the virus can be thermally stable, or at least the virus has improved thermal stability as compared with a native virus. In some examples, the particle is stable after heating at 90° C. for at least 5 minutes, or at least 10 minutes, or at least 20 minutes, or at least 30 minutes. In some examples, the particle is stable after storage at 21° C. for at least 24 hours, or at least 48 hours, or at least 1 week, or at least 1 month, or at least 3 months, or at least a year. In some examples, the particle is stable after storage at 4° C. for at least 1 week, or at least 1 month, or at least 3 months, or at least a year. The measurement of stability can be determined by plaque assay, i.e., wherein after release, the virus is substantially intact and substantially functional to replicate and/or form a plaque.

In the particle disclosed herein, the virus may be stable to acid or base. An particle disclosed herein can be subjected to low pH such as ≤pH 4.0, ≤pH 3.5 or ≤pH 3.0. When subsequently released from particle, the virus is substantially intact and substantially functional. By contrast a virus that is not ensilicated is more likely to be denatured and lose its function in these conditions.

### Method

Disclosed herein is a method of producing a virus encapsulated in an amorphous silica shell, the method comprising enriching or purifying a virus and suspending the virus in buffer, hydrolysing a silica precursor and directly contacting the hydrolysed silica precursor about the surface of the virus in buffer to encapsulate the virus in an amorphous silica shell.

The method can be used to form the particles described herein. The virus may be any suitable virus as described herein. The amorphous silica shell may be as otherwise described herein.

In some embodiments, the method further comprises a step of propagating the virus prior to enriching or purifying the virus. In some embodiments, the propagating is solid-phase or liquid-phase propagation. The propagating comprises culturing the virus in the presence of bacteria in growth buffer. In some examples, the growth buffer is or comprises tryptic soy broth (TSB) (i.e., including modified TSB buffers), Luria Broth (LB), Super Optimal broth with Catabolite repression (SOC), Super Optimal Broth (SOB), Terrific Broth (TB), YT broth (2× YT Broth).

The method comprises an enriching or purifying step. The enriching or purifying step may be via any suitable enrichment or purification method. In some embodiments, the enriching or purifying is selected from a PEG-based enrichment, gradient centrifugation, size exclusion chromatography, ion exchange chromatography chloroform purification, or filtration methods. In preferred embodiments, the enriching or purifying is from a virus in a lysate (i.e., virus in growth buffer). In preferred embodiments, the enriching or purifying is a PEG-based enrichment, where PEG is polyethylene glycol. In some examples, the PEG has an average molecular weight between 3000 and 12000 g/mol, or between 3500 and 10000 g/mol, or between 4000 and 80000 g/mol, or between 5000 and 7000 g/mol, or about 6000 g/mol. In some examples, the PEG-based enrichment comprises PEG 6000, i.e., PEG with an average molecular weight of 6000 g/mol. In some examples, the PEG-based enrichment comprises adding between 1 and 15% w/w, or from 5 to 12.5% w/w, or from 7 to 10% w/w PEG to the virus (i.e., virus lysate). In some examples, the PEG-based enrichment comprises adding at least 5% w/w, or at least 7% w/w, or at least 10% w/w PEG to a virus (e.g., virus lysate). In some embodiments, the enriching or purifying comprises a chloroform purification.

In some embodiments, the enriching or purifying comprises gradient centrifugation. In some embodiments, the gradient centrifugation utilizes a gradient medium selected from sucrose, cesium chloride, iodixanol, sorbitol, Histodenz, or Dextran. In some embodiments, the enriching or purification comprises size exclusion or ion exchange chromatography. In some embodiments, the enriching or purifying comprises filtration methods.

The enriched or purified virus may added to any suitable buffer to form an enriched virus in buffer. In preferred embodiments, the enriched or purified virus is suspended in buffer. In some embodiments, the buffer comprises one or more salts, and preferably wherein the one or more salts comprises a dicationic or monocationic metal ion. In some embodiments, the one or more salts may comprise a dicationic metal ion. In some embodiments, the one or more salts may comprise one or more of magnesium or calcium. In some embodiments, the one or more salts may comprise a monocationic metal ion, wherein the monocationic metal ion may comprise sodium. In some examples, the one or more salts may comprise magnesium sulfate and/or sodium chloride. In some embodiments, the buffer comprises one or more dicationic metal salts at a concentration of between 0.1 mM and 50 mM, or between about 1 mM to 25 mM, or between about 5 mM to 11 mM, or about 8 mM. In some embodiments, the buffer comprises one or more dicationic metal salts at a concentration of greater than 0.5 mM, or greater than 1 mM, or greater than 2 mM, or greater than 3 mM, or greater than 4 mM, or greater than 5 mM, or greater than 6 mM, or greater than 7 mM, or greater than about 8 mM. In some embodiments, the dicationic metal salt preferably comprises magnesium and more preferably comprises magnesium sulfate.

In some embodiments, the buffer comprises a sodium salt at a concentration of from 0.1 mM to 500 mM, or between about 50 mM to 200 mM, or about 100 mM. In some embodiments, the buffer comprises one or more dicationic metal salts at a concentration of greater than 0.1 mM, or greater than 1 mM, or greater than 5 mM, or greater than 10 mM, or greater than 25 mM, or greater than 50 mM, or greater than 75 mM, or about 100 mM. In preferred embodiments, the sodium salt is preferably sodium chloride.

In some embodiments, the buffer may comprise one or more small molecules. In some embodiments, the buffer comprises one or more amino acids. In some embodiments, the buffer comprises glycine. In some embodiments, the buffer comprises an amino acid such as glycine at a concentration of at least 1 mM, or at least 5 mM, or at least 10 mM, or at least 25 mM, or at least or equal to 50 mM. In some embodiments, the buffer comprises glycine at a concentration of between 1 mM and 100 mM, or between about 10 mM to 80 mM, or between about 25 mM to 75 mM, or between about 40 mM to 60 mM, or about 50 mM.

In some embodiments, the buffer may comprise one or more stabilizers. In some embodiments, the buffer may comprise gelatin. In some embodiments, the buffer may comprise at least 0.01 g/mL of gelatin, or at least 0.5 g/mL, or about 0.1 g/mL of gelatin.

In some embodiments, the buffer comprises or is SM buffer, a modified SM buffer (e.g., glycine-modified SM buffer), PBS buffer, imidazole buffer, bis-tris buffer or a sucrose buffer. In some examples, the buffer is not a Tris buffer (i.e., a buffer comprising Trizma and water alone). In some examples, the buffer is free of Trizma.

In certain embodiments, after the purifying and enriching, the virus in buffer has a polydispersity index (PDI) of less than 0.2, more preferably less than 0.15, or less than 0.14, or less than 0.13, or less than 0.12 as determined using DLS analysis. In some examples, the virus in buffer has a PDI of between 0.1 and 0.15.

In preferred embodiments, the enriching comprises concentrating the concentration of virus (i.e., wherein the virus in buffer has increased concentration or PFU/mL). In some embodiments, the concentrating comprises centrifuging the virus. The centrifuging may take place at any suitable speed, for example, from 5,000×G to 200,000×G. In some embodiments, for example, for phage K, the centrifuging takes place at a speed of less than 25000×g, or preferably less than 16000×g.

In preferred embodiments, after enriching and purifying, the virus in buffer (i.e., the enriched virus in buffer) is present at a concentration greater than 1×10⁷ PFU/ml, more preferably greater than 1×10⁸ PFU/ml, more preferably greater than 1×10⁹ PFU/ml, more preferably in a range of from 1×10⁸ PFU/ml to 1×10¹⁰ PFU/ml, or from 1×10⁹ PFU/ml to 1×10¹⁰ PFU/ml.

The method of the present invention also comprises a hydrolysis step, which comprises hydrolysing a silica precursor. The resultant product is a hydrolysed silica precursor, which may comprise a silica coordinated by four oxygen atoms. In preferred embodiments, the hydrolysed silica precursor is a silicic acid monomer, e.g., of the formula e.g., Si(OH)₄. The pre-hydrolysing step preferably occurs in the absence of virus. In some embodiments, the pre-hydrolysing step is carried out until the solution becomes monophasic. In some embodiments, the pre-hydrolysis step is or occurs for about 20 to 120 minutes, or about 40 to 80 minutes. In some embodiments, the pre-hydrolysis step is or occurs for at least 20 minutes, or at least 40 minutes, or for at least or equal to 60 minutes. In some embodiments, the pre-hydrolysis step is or occurs for less than 120 minutes, or less than 90 minutes, or less than or equal to 60 minutes.

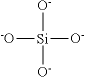

The silica precursor may be any suitable silica precursor, i.e., any molecule that hydrolyses to form silica, more preferably a silica monomer as described above. In some embodiments, the silica is covalently attached to one or more alkoxide groups (i.e., the silica precursor is an alkoxysilane). In some embodiments, the silica precursor is a tetra-alkyl orthosilicate. In some embodiments, the alkyl of the tetra-alkyl orthosilicate is a C₁-C₆ alkyl. Examples of silica starting materials include tetra-methoxy-orthosilicate (TMOS), tetra-ethoxy-orthosilicate (TEOS), tetra-propoxy-orthosilicate (TPOS), tetra-butoxy-orthosilicate (TBOS), and tetra (ethoxymethoxy) silane. In some embodiments, the tetra-alkyl orthosilicate comprises or is tetra-ethyl orthosilicate.

Hydrolysing a silica starting material is typically performed at acidic pH. The acidic pH can be less than or equal to 4.5, 3.5, 3.0, 2.5 or 2.0. In some examples, the step of hydrolysing the silica precursor is performed at pH 3.0 or below. A preferred acidifying agent is HCl, e.g. 32% HCl. The acidifying agent catalyses the hydrolysis of TEOS. In some examples, a 1:1 ratio of tetraethyl orthosilicate solution to HCl is used.

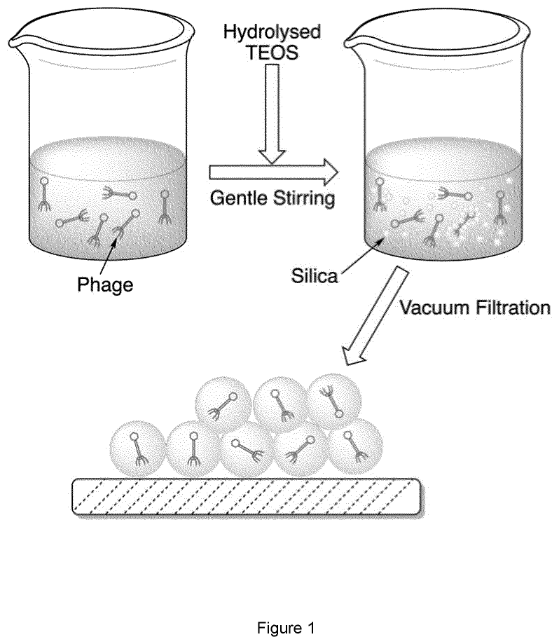

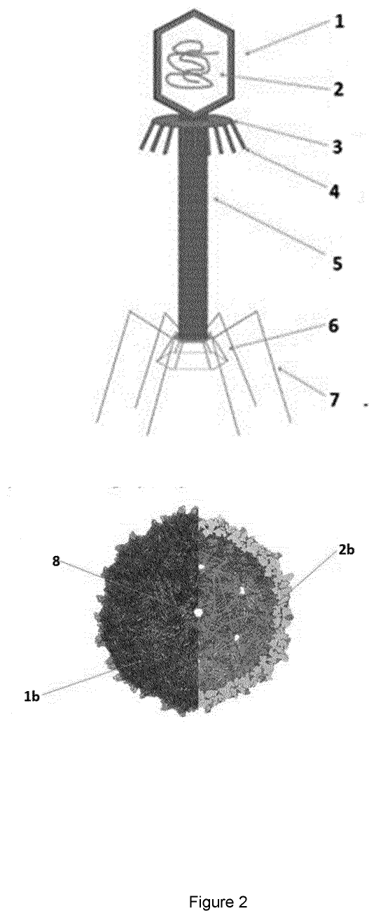

The method further comprises directly contacting the hydrolysed silica precursor with the surface of the virus in buffer to encapsulate the virus in an amorphous silica shell. In some embodiments, the contacting comprises adding the hydrolysed silica to the virus in buffer, and preferably stirring the mixture. A schematic of this process is shown in FIG. 1. In this step, the hydrolysed silica precursor is preferably added to the virus in buffer. In some embodiments, the hydrolysed silica precursor (i.e., silicic acid monomers) first interacts with the surface of the virus (e.g., by electrostatic interaction between the silica and the protein capsid) and the silica shell then grows and polymerises about the surface of the virus. This may otherwise be referred to as ensilication about the virus, and the resultant particle may be referred to as an ensilicated virus.

The encapsulation step to form a silica shell about the surface of the virus is different from a typical sol-gel process in a number of aspects. Ensilication starts with silicic acid monomers, which when added to the virus in buffer, polymerise about the surface of the virus itself. In particular, the hydrolysed silica precursor is typically attracted to the positive residues on the surface of the virus, and electrostatically bind to the virus surface. Subsequent silica monomers then join the silica chain leading to growth of a silica shell about the virus surface. The present methods can be contrasted with previous work comprising silica coating of viruses which use typical sol-gel processes where instead a polymerised silica network is formed prior to contact with the biomolecule. Different to previous silica encapsulation methods, the silica is instead deposited about the surface of the virus as opposed to deposition about a template or intermediate structure formed around the virus (e.g., using a polycationic polymer). The method of the present invention can be free of a templating step, e.g., wherein an intermediate layer is formed around the virus using a polycationic polymer such that the silica is not in direct contact with the surface of the virus. In some embodiments, the method may be free of polycationic polymer. In some embodiments, the method may be free of polylysine.

The contacting can be carried out at a pH>6. In preferred embodiments, the contacting carried out at a pH greater than 7, or greater than 7.25, preferably wherein the pH is between 7.25 and 8.5, for example, about 7.5.

The contacting may be carried out for any suitable time. In some embodiments, the contacting is carried out for at least 5 minutes, preferably at least 10 minutes. In some embodiments, the contacting is carried out for less than 60 minutes, or less than 45 minutes, or less than 20 minutes, or less than 10 minutes, or less than 5 minutes, or less than 2 minutes, or less than 1 minute, or less than 30 seconds. In some embodiments, the contacting is carried out for between 10 and 20 minutes.

The contacting step may be carried out using any suitable ratio of hydrolysed silica prescursor:virus in buffer. In some embodiments, the ratio of hydrolysed silica prescursor:virus in buffer is from 1:1 to 1:250, more preferably from 1:10 to 1:250. In preferred embodiments, the ratio of hydrolysed silica to the virus in buffer is from about 1:75 to 1:150, for example, about 1:100. In some embodiments, the ratio of hydrolysed silica to the virus in buffer 1:≥10, or 1:≥25, or 1:≥50, or 1:≥75, or 1: ≥100.

The contacting step may be carried out at any suitable temperature. In some embodiments, the contacting may be carried out at a temperature between 4 and 40° C., or between 4 and 25° C., or between 15 and 25° C., or between 15 and 20° C.

In some embodiments, the resultant particles precipitate out. In some embodiments, the method further comprises a vacuum filtering step, i.e., wherein the particles are vacuum filtered. In some embodiments, the particles may be further subjected to drying, e.g., air-drying.

### Dry Powder

The present invention also provides for a dry powder comprising a particle of the present invention, more preferably a plurality of particles of the present invention. The dry powder may be obtained by the method described in the second aspect, followed by vacuum filtration and drying. The dry powder may comprise at least one first particle comprising a first virus and at least one second particle comprising a virus different from the virus in the first particle (i.e., a second virus).

### Pharmaceutical Composition

The present invention also provides for pharmaceutical composition comprising a one or more particles of the present invention, more preferably a plurality of particles of the present invention. In some embodiments, the particle of the present invention may be in the form of a dry powder as described above. The pharmaceutical composition may comprise at least one first particle comprising a first virus and at least one second particle comprising a virus different from the virus in the first particle (i.e., a second virus). The pharmaceutical composition may otherwise be referred to as a composition.

The pharmaceutical composition may further comprise one or more pharmaceutical excipients or adjuvants.

In some embodiments, the one or more excipients may be selected from emulsifiers, osmolytes, co-solvents, antimicrobials or a combination thereof. Examples of emulsifiers may be Tween 20 or Tween 80. Examples of osmolytes include sucrose. Examples of co-solvents may be sorbitol and/or glycerol. Examples of antimicrobials include phenol and/or benzethonium chloride). In some embodiments, the particle may comprise one or more adjuvants. In some embodiments, the adjuvants may be selected from alum, a monophosphoryl lipid (e.g., MPLA), a squalene based-adjuvant (e.g., AS03 or MF59), AS04 (e.g., a combination of MPLA and alum), or CpG DNA adjuvant (e.g., CpG 1018).

### Therapy

The particle described herein may be for use in therapy. The particle described herein may be used in a method of treatment or prevention of a disease. In some embodiments, wherein the virus is a bacteriophage, the virus may be for use as an antibacterial medicament (i.e., phage therapy). In some embodiments, wherein the virus is a mammalian virus (e.g., a live attenuated virus or an inactivated virus), the virus may be for use as a vaccine, i.e., to provide protective immunity against infection, for example, wherein the infection is a bacterial infection or a viral infection. In some embodiments, the particle for use is administered by any suitable means, e.g., intramuscularly, subcutaneously, topically, intranasally or orally.

### Non-Therapeutic Use

Also disclosed herein, is the non-therapeutic use of the particles, powder or pharmaceutical composition described herein, for use as an antimicrobial, wherein the virus is a bacteriophage. In some examples, the particle described herein may be used as an antimicrobial wherein the particles are applied to a surface, (i.e., wherein the bacteriophage is preserved on the surface). In some embodiments, the surface may include, but is not limited to, the surface of a structure, a packaging material, a tool or piece of equipment (e.g., a medical implement), or a biological material, such as plant matter. This can make that surface inimical to bacterial growth.

### Method of Release

Also disclosed herein is a method of releasing the virus from the particle of the present invention.

The virus may be released prior to its use for administration to the subject, e.g., prior to injection. In some embodiments, after release, the virus may be mixed with a further adjuvant or pharmaceutical carrier prior to administration to the virus.

In some embodiments, the method comprises contacting the silica shell with a solution (i.e., a release solution) comprising fluoride ions at acidic pH (i.e., to produce HF). The solution may comprise sodium fluoride, which is acidified, for example, using HCl. The method may further comprise agitation of the mixture. In some embodiments, the contacting may be from about 10 minutes to about 2 hours, or from 30 minutes to about 90 minutes The solution may have a pH of less than 6, or less than 5, or less than 4, or less than 3, in some examples, about 2.9. The HF concentration in the solution is preferably less than or equal to 3 mg/mL, or less than or equal to 2.5 mg/mL, or less than or equal to 2 mg/mL, or less than or equal to 1.5 mg/mL, or less than or equal to 1 mg/mL, or less than or equal to 0.5 mg/mL. In some embodiments, the total concentration of HF in contact with the particle is less than 0.5 mg/mL. In some examples, the solution comprises SM buffer or water (i.e., deionized water).

In other embodiments, the virus can be released in a method without contacting the particle with fluoride ions at acidic pH (e.g., for an isometric virus such as MS2). In some embodiments, the virus is released by incubating the particles (e.g., in growth buffer at 37° C.)

### EXAMPLES

### Materials and Methods

### Materials

Growth media tryptic soy agar (TSA) and tryptic soy broth (TSB), TEOS, HCl, NaF, Trizma base, and chloroform were purchased from Sigma Aldrich.

### Solid Phage Propagation

### Phage K

Bacteriophage K and Staphylococcus aureus (MSSA H560) host strains were kindly provided by Dr S. Milo of the Jenkins group at University of Bath. A single colony from the provided MSSA streak plate was placed into 10 mL of tryptic soy broth (TSB) solution (Sigma Aldrich) and incubated at 37° C. with shaking overnight. Approximately 15 mL of tryptic soy agarose (TSA) gel was poured onto sterilin 90 mm round Petri dishes to form bases for bacterial growth. 100 μL of the bacterial overnight solution and 100 μL of provided phage lysate was added to 3 mL of 0.7% TSA containing TSB solution to make soft top agar. 3 mL of this soft agar was poured on to TSA gel dishes and allowed to set before being incubated for 18 hours at 37° C. statically. After incubation 3 mL of SM buffer (pH 7.5) was added to each dish where upon the dishes were incubated for a further 4 hours under previous conditions. After incubation the free SM buffer approximately 3 mL per plate, was pipetted into a centrifuge tube and spun at 10,000 RPM at 4° C. for 10 minutes. The top layer was taken and filtered through at 0.22 μm millex filter and collected to be stored at 4° C.

### MS2

Protocol provided by ATCC with phage and host strain E. coli 15597. Bacterial cultures were defrosted at room temperature and a streak plate conducted on a clean TSA petri dish. This plate was incubated over night at 37° C. Once fully grown, one colony was selected from this plate, placed in 10 mL of TSB to form a bacterial overnight solution, and incubated at 37° C. for 18 hours.

1 mL of this overnight culture was pipetted into the lyophilised phage sample and carefully inverted to rehydrate the phage. Approximately 15 mL of tryptic soy agarose (TSA) gel was poured onto Sterilin 90 mm round Petri dishes to form bases for bacterial growth. 100 μL of the overnight solution and 100 μL of provided phage lysate was added to 3 mL of 0.7% TSA containing TSB solution to make soft top agar. 3 mL of this soft agar was poured on to TSA gel dishes and allowed to set before being incubated for 18 hours at 37° C. statically. After incubation 3 mL of SM buffer (pH 7.5) was added to each dish whereupon the dishes were incubated for a further 4 hours. After incubation the free SM buffer approximately 3 mL per plate, was pipetted into a centrifuge tube and spun at 10,000 RPM at 4° C. for 10 minutes. The top layer was taken and filtered through at 0.22 μm Millex filter and collected to be stored at 4° C.

### Small-Scale Enrichment of Bacteriophage K

10 mL of impure phage lysate was centrifuged at 15,300×g at 3° C. for 20 minutes, slow acceleration, no mechanical deceleration (Beckman Coulter Avanti J-26S with JA-25-50 rotor). The supernatant solution was then added to a vessel in an ice bath on top of a magnetic stirrer. NaCl (Sigma-Aldrich) was slowly added under gentle stirring until the final concentration reached 2.3% w/w. Polyethylene glycol M/W 6000 (PEG-6000) was subsequently added under gentle stirring until finally concentration reached % w/w. While maintaining the gentle stirring the beaker was covered with aluminium foil and left for 1 hour, after which the ice bath was removed from stirring and placed in a refrigerator at 4° C. overnight. The chilled phage lysate was then centrifuged as before at 15,300×g at 3° C. for 20 minutes, slow acceleration, no mechanical deceleration (Beckman Coulter Avanti J-26S with JA-25-50 rotor). The supernatant was discarded, and the precipitate re-suspended in a small volume of SM buffer (pH 7.5). The suspension was then transferred to a clean centrifuge bottle and the previous bottles rinsed, with the washings added to the suspension in the ‘clean’ bottle. The suspension was then further centrifuged at 13,000×g for 4 minutes at 23° C., slow acceleration, no mechanical deceleration, to promote precipitation of the PEG-6000 (Beckman Coulter Avanti J-26S with JA-25-50 rotor). The supernatant was collected and stored at 4° C. for later use.

### Liquid Phase Propagation of Bacteriophage K

30 mL of TSB was inoculated with bacterial freezer stocks, and left to incubate with shaking at 37° C., 180 RPM, for approximately 18 hours, to produce a bacterial overnight solution. 10 mL of this overnight culture was added to each of 3× 600 mL medic flasks containing 400 mL fresh TSBss. The medic flasks were incubated, with shaking, at 37° C., 170 RPM, for exactly 18 hours. The contents of the flasks were then centrifuged at 15,300×g, 3° C. for 20 minutes (Beckman Coulter Allegra 64R with TA-10-250 rotor) and the supernatant retained.

### Large Scale Enrichment of Phage

The supernatant collected from a liquid phase propagation was transferred to a 2 L plastic beaker in an ice bath a-top a magnetic stirrer. NaCl was gradually added, with constant gentle stirring (approx. 150 RPM) to a final concentration of 2.3% w/w. Once dissolved PEG-6000 was added, again under gentle stirring, to a final concentration of 7% w/w for phage k or 10% w/w for the optimised MS2 method. The beaker was then covered with foil and left to stir for 1 hour before being transferred to a cold room at 4° C. overnight. Any precipitate was discarded, and the solution was then centrifuged at 15,300×g, 3° C., for 20 minutes (K) or 40 minutes (MS2), slow acceleration (K), no mechanical deceleration (K), or rapid acceleration (MS2), and a high degree of mechanical deceleration (MS2) (Beckman Coulter Allegra 64R with TA-10-250 rotor). The supernatant was discarded and the pellets resuspended in SM buffer to bring the total volume of resuspension to be 150 mL. For phage k, the suspension was then centrifuged at 13,000×g, 23° C., for 4 minutes, slow acceleration, no mechanical deceleration (Beckman Coulter Avanti J-26S with JA-25-50 rotor), after which the supernatant was transferred to long term storage vessels. For MS2, the phage is centrifuged for 40 minutes with higher acceleration and deceleration as detailed above.

### Chloroform Purification of MS2

To 40 mL of enriched MS2 lysates an equal volume of CHCl₃ is added. The mixture is vortex mixed and subsequently centrifuged at 3000×G for 15 minutes. The mixture should then be separated into 3 distinct layers. The top aqueous layer is collected using a pipette and transferred to long term storage vessels at 4-8° C.

### Ensilication of Phage

Preparation of prehydrolysed silica: 40 mL of a 1:1 mixture of tetraethyl orthosilicate (TEOS) and H₂O, along with 40 μL of 32% HCl was stirred at 700 RPM for approximately 40 minutes until the solution became monophasic, after which stirring speed was reduced to 125 RPM for 20 minutes. This solution is the prehydrolysed silica solution.

In a separate beaker 90 mL of SM buffer for phage K or SM(glycine) buffer for MS2 and 10 mL of enriched high titre (>1.00×10⁹ PFU/mL) phage lysates were mixed and gently stirred at 125 RPM to ensure they are fully mixed. 1 mL of PHOS is added to the phage suspension and the mixture stirred at 125 RPM for 10 minutes. After this time the mixture is filtered under vacuum through 0.7 μm micro-glass fibre filters (Fisher Scientific). The filter and material is left to dry in fume hood until a dry powdery substance is obtained. The powder is then removed from the filter and placed in an Eppendorf vial for long term storage.

### Plaque Assay

10 mL bacterial overnight solution was created. 100 μL/dish was added to 3 mL/dish of warm TSB containing 0.7% (w/v) TSA and left to stand for 10 minutes. 3 mL of the inoculated 0.7% TSA solution was added to each TS agar plate and left to set in sterile conditions. 100 μL of phage lysate was added to 900 μL SM buffer giving a 10× dilution, this step was serially repeated to produce a dilution range of 10-1-10-9. 10 μL of each dilution was pipetted, in triplicate, onto agar dishes with 3 different dilutions per dish. The spots were left to dry in sterile conditions before the dishes were incubated (37° C., 18 hours). Once incubated the visible plaques were counted to obtain a plaque forming unit per millilitre (PFU/mL) titre amount.

Release of Ensilicated Phage with HF

A 10 mg of ensilicated powder was placed into 1 mg/mL solution of NaF in SM buffer, pH 2.90. This was spun head over heels for 1 hour before 2 mL of this solution was transferred to a 40 mL culture of 1-hour old bacteria. This was then incubated for 24 hours at 37° C., 170 RPM. After which this was filtered through a 0.45 μm filter before being stored at 4° C. ready for assay.

In optimisation experiments of NaF/HF release, a 96 well plate enriched phage lysates suspended in SM buffer were mixed with gradually increasing concentrations of NaF/HF buffer. This mixture was then added to an 18-hour old bacterial culture, in TSB, and the optical density (measured at 595 nm) was taken to determine the presence of bacterial growth, and therefore the survival of the phage.

Release of MS2 Ensilicated Phage without HF

A small mass (40 mg) of ensilicated MS2 powders were added to 1 mL of TSBss and incubated for 1 hour before being added to 400 mL of 1-hour old E. coli and incubated for a further 24 hours. These samples were then plaque assayed.

### Results and Discussion

### Enrichment/Purification of Phage

### Phage K

Initial attempts to silica encapsulate the native phage K lysates were unsuccessful. Using 10:1 of lysates to prehydrolysed silica, in 90 mL of SM buffer, solutions were mixed for 10 minutes, there was no turbidity or particle formation. Further stirring still resulted in no change and no visible formation of silica particles. It was eventually found that only properly enriched and purified phage lysates were suitable for ensilication. Ensilication of an enriched phage lysate resulted in almost instant turbidity and the formation of silica nanoparticles in solution. The results of these tests showed that only properly enriched phage lysates are suitable for this ensilication process.

The phage lysate was enriched using an enrichment method as described above using PEG 6000, although chloroform purification may also be used to purify the lysate. The PEG6000 enrichment method also importantly reduced the volume of the lysates by a factor of 10 which had the benefit of increasing the average PFU/mL of enriched lysates by 10×. The PEG-enrichment method removed much of the undesired bacterial debris and centrifuging and resuspending the subsequent mixture in SM buffer effectively removed the TS broth.

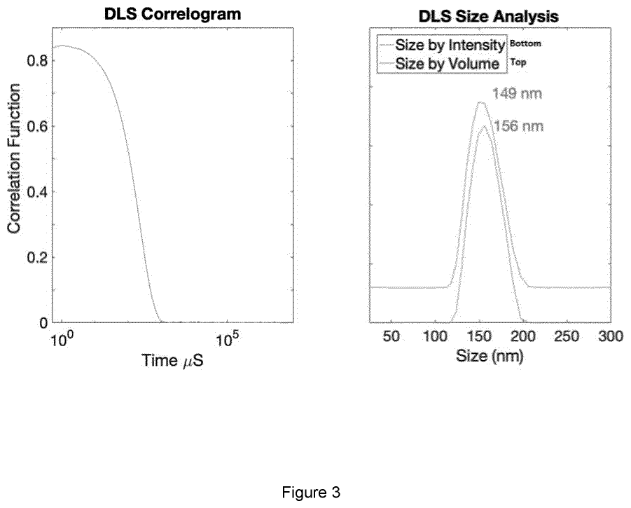

The enrichment bacteriophage K utilising PEG 6000 enabled lysates to be obtained with increased purity for further study and experimentation. After enrichment DLS analysis indicated that the enriched solution had a much lower polydispersity as compared to non-enriched solutions, with a calculated PDI of 0.118 as compared to an unenriched solution which had a PDI of 0.384. After enrichment, the correlogram obtained from DLS analysis showed a rapid initial decline, at approximately 10 pS, in signal correlation followed by a sharp slope with no peak shoulders (see FIG. 3). These results are indicative of a relatively small particle size with a highly mono-dispersed distribution and no impurities.

With the successful confirmation of the enrichment processes, the enriched lysate was subjected to an identical plaque assay process to that of the impure lysate obtaining a titre of 5.4×10² PFU/mL±11%, approximately 1 log higher than the impure lysate.

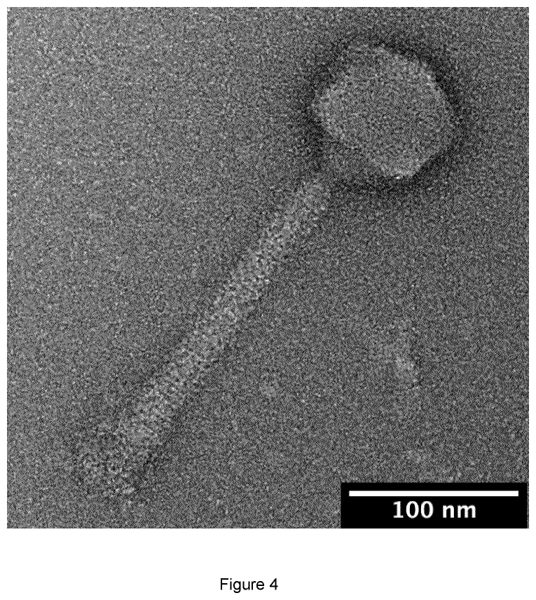

During optimisation of the enrichment process for phage K, it was found that high acceleration and deceleration within the centrifugation process could lead to mechanical and shear stress on the phages, with faster centrifugation speeds leading to enriched lysates with titre values lower than in the optimised method as indicated above. TEM analysis of these enriched solutions showed that some of the phage virions had missing or fractured heads causing premature ejection of the genetic material. Reducing the centrifugation speed and removing the assistive deceleration in line with the optimised enrichment process as indicated above was found to help alleviate the mechanical and shear stress on the phages. TEM analysis of these samples showed very few broken or spent phages with large amounts of intact virions (see FIG. 4).

### MS2

The enrichment and purification methods developed for bacteriophage K could also be readily applied to MS2, however, the above methods produced a volume of lysate that was of lower titre as compared to the results for phage K. To increase further the titre value of the phage lysates, a larger amount of PEG 6000 was added to the enrichment step for MS2 as compared with phage K (i.e., 10% w/w as opposed to 7% w/w).This may be because MS2 is smaller, and so a higher level of PEG is more effective to force the virions to salt or crystallise out of solution. Further, a harsher centrifugation step was used for MS2 after PEG enrichment as compared to phage K. Implementing a longer and harsher centrifugation step is believed to be less damaging for MS2 due to its simpler structure. After this optimisation, plaque assays of lysates resulting from these two changes were at 3.50×10¹⁰ PFU/ml. TEM analysis of enriched MS2 showed a large amount of spherical values, with no sign of spent or broke phage capsids.

### Further Purification of MS2 Using Chloroform

The enriched MS2 lysate was purified further. To the enriched MS2 lysate mixed with an equal volume of chloroform before being vortexed to ensure that the two solutions were sufficiently mixed. This was followed by a low speed centrifugation step. Plaque assays and DLS analysis of these purified solutions were carried out. The titre of these purified solutions was 3.7×10⁷ PFU/ml. Therefore, with an extra purification step, DLS analysis saw only a very minimal increase in purity. This indicates a second or additional purification step is not needed.

### Silica Encapsulation/Ensilication of Phage K

A 1:100 ratio of prehydrolysed silica to buffer was used, the enriched phage sample was ensilicated for 10 minutes before being vacuum filtered through glass microfibre paper and air dried over 48 hours. A schematic of the silica encapsulation is shown in FIG. 1. Batch ensilications of 10 mL of 7.47×10⁹ PFU/mL of enriched phage lysate typically produced 159.3 mg±55.6 mg (2× SD) of ensilicated powder. BCA protein assays showed that 1 mL of the same titre phage lysate gave a 1.33 mg/mL protein concentration.

In addition to the required enrichment step, the method itself was also further modified as compared to silica encapsulation previously used for small polypeptides to improve the encapsulation process. A higher pH of 7.5 was used well as the replacement of Tris buffer with sterilised SM buffer for phage K, or a modified SM buffer comprising glycine for phage MS2. NaCl and MgSO₄ salts form a major component of the SM buffer.

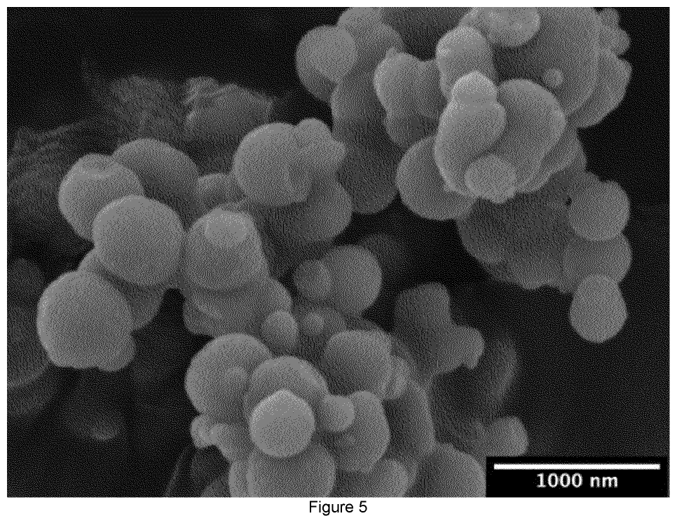

The images collected of ensilicated phage K showed a highly networked structure comprised of individual spheres (see FIG. 5). The spheres themselves had a median size larger than that of previously silica encapsulated biomolecules. The average particle size was found to have a median of around 400 nm, as determined by FE-SEM imaging. Furthermore, the visible surface of the sphere appeared much smoother than anticipated. The silica shell was also found porous (see further discussion below).

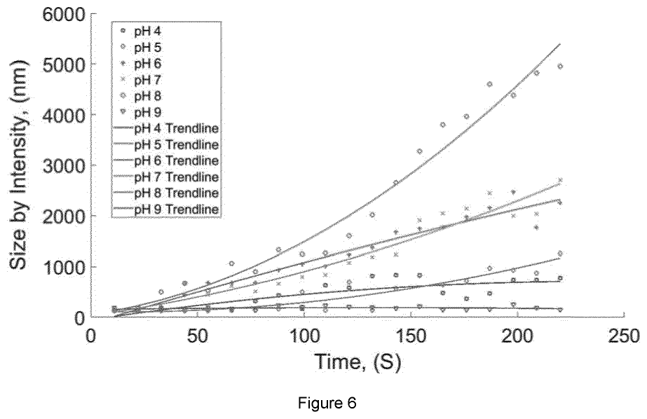

The increase of pH was found to be a contributing factor to the speed of ensilication. Investigations into the effect of pH on the rate of ensilication showed an increase of pH caused an increase in reaction rate until pH 9.0 was reached, where the net charge of solution prevented the formation of silica monomers required for the reaction (see FIG. 6). The increased solubility of silica at higher pH is believed also to contribute to particles growing to a larger size. Furthermore, the buffer composition used in ensilication was found to be important, with the selection of the buffer affecting the recovery rate of the phage. It is believed that the addition of double cationic salts (e.g., salts comprising Mg²⁺ or Ca²⁺) is important for the propagation of bacteriophages and the absence of these salts reduced the titre of propagated phage solutions. Further, it is believed that the presence of sodium salts may contribute to the stability of liquid lysates. In particular, the sodium salts can prevent aggregation as well as relieving hydrostatic pressure that could be fatal to the virus or phage capsid. The presence of sodium ions was found to promote particle growth, causing final silica nanoparticles to be of a larger size after 10 minutes as compared to those created without sodium ions present. Overall, it was found that addition of salt at a lightly basic pH (e.g., SM buffer) created an environment that is more suitable for particle size growth than rapid aggregation and precipitation.

The concentration of phages in the lysate was also deemed to be important. Phage lysates used for ensilication with >1×10⁷ PFU/mL were found to contribute to more successful ensilication and release, resulting in the highest surviving release titres.

Particular ratios of phage lysates to prehydrolysed silica was also found to be optimum. For higher ratios of lysates to silica, the ensilication process is believed to be less uniform resulting in a larger portion of the phage virions left almost untouched, with other virions being fully ensilicated.

### Ensilication of MS2

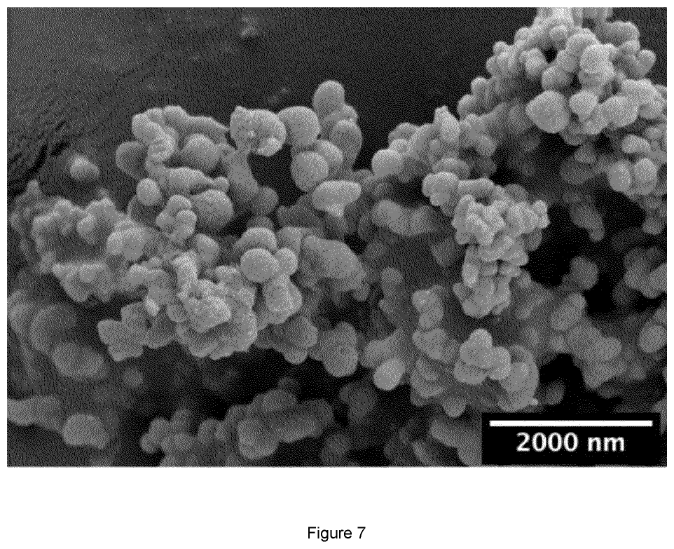

The ensilication for MS2 was modified from that described for phage K in that a modified SM buffer was used wherein 50 mM Trizma base was exchanged for 50 mM of glycine. This was found to improve the electrostatics between the silica and the phage which promoted the ensilication process. Ensilication with this buffer produced turbidity after a few minutes stirring and a large number of silica nanoparticles were produced after several minutes of stirring. Ensilication of MS2 using SM(G) buffer produced an average of 146.25 mg (±3.46%) of powder. FE-SEM imaging of the powders are shown in FIG. 7. The appearance of the particles are similar to those seen for phage K. Individual spherical particles form chains that combine into characteristic silica networks. The individual spheres measure between 300-500 nm in diameter and the morphology of the spheres is less uniform than phage K, with several particles having an ellipsoid shape. The surface of the particles are also different compared to phage K particles. MS2 indicate a significantly more porous appearance as compared to phage K.

BCA assays of MS2 powders revealed that the powder comprised 17.5±1.9% protein and 82.5±1.9% silica.

Release of Bacteriophage K from Silica Shell

### HF Release

A low concentration hydrogen fluoride buffer can be used to dissolve the silica shell around the phages, without causing significant damage to the product.

Bacteriophage K was found to be more sensitive to the weak acid than previously studied proteins and as such the concentration of NaF/HF buffer used for release were optimised.

As the concentration of HF used in protein studies is 4 mg/mL a dilution of 7.91× gives a tolerance of the phage to 0.51 mg/mL HF. An increase in the acid concentration above this value resulted in destruction of the phage super structure.

However, the silica coating the phages are preferentially digested by the acid, meaning the concentration of the release solution would need to contain sufficient acid to totally dissolve the silica shells with an excess concentration of less than 0.51 mg/mL. Table 1 shows the experimental results of releasing samples of ensilicated phage K with various concentrations of HF and time scales of bacterial incubation. The use of 2 mg/mL of HF in the release of approximately 10 mg of 8.66×10⁹ PFU/mL ensilicated phage was the highest concentration of HF tolerated by the ensilicated phages. Using higher concentrations of HF or lower titre powders resulted in the death of ensilicated phages.

TABLE 1
Table showing details plaque assay results
from ensilicated and released phage K.

| | Starting | Amount | | |
|---|---|---|---|---|
| | Titre | of | | |
| | Before | Powder | HF | Plaque Assay Titre |
| | Ensilication | Released | Concentration | After Ensilication |
| Sample | PFU/mL | mg | mg/mL | PFU/mL |
| 1 | 6.33 × 10⁹ | 15.2 | 1 mg/ml | 800 |
| 2 | 6.33 × 10⁹ | 16.0 | 2 mg/ml | 2333 |
| 3 | 6.33 × 10⁹ | 15.7 | 3 mg/ml | 0 |
| 4 | 6.33 × 10⁹ | 15.3 | 4 mg/ml | 0 |
| 5 | 8.66 × 10⁹ | 20.0 | 1 mg/ml | 66 |
| 6 | 8.66 × 10⁹ | 9.4 | 1 mg/ml | 333 |
| 7 | 8.66 × 10⁹ | 10.1 | 2 mg/ml | 200 |
| 8* | 8.66 × 10⁹ | 21.3 | 1 mg/ml | 3660 |
| 9 | 4.36 × 10⁸ | 10.1 | 1 mg/ml | 966 |

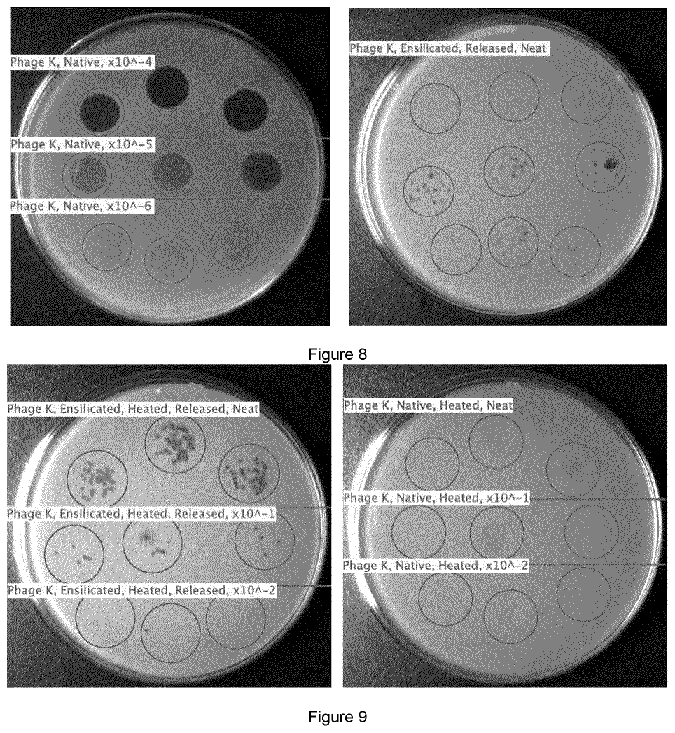

The above results suggest that released samples can be used to form a plaque forming unit even after ensilication and subsequent release. The most successful released samples represent a plaque forming unit loss of approximately 6 log. This decrease, while appearing large, is importantly of little overall concern to the success of bacteriophages. This is because only a small number of phages are required to grow a phage population back to high titre values of ×10⁸ or more. The number of surviving phages in the ensilicated, and released bacteriophage K powders is therefore more than enough for this regrowth to take place. Among these was sample 9, this sample was stored at ambient conditions during its 18-month (days) life with temperatures ranging from approximately 10-35° C. Images of this plaque assay are shown below in FIG. 8. The presence of a dark spot on the bright bacterial lawn represents a single ‘plaque forming unit’ or phage virion. Counting these individual spots and factoring in sample volume and dilution can give a value for the infections phage particles in a given volume, quoted as PFU/mL. PFU/mL for native unensilicated phage lysates can be compared to PFU/mL obtained from ensilicated and released phage to show the rate of phage survival during the process. The growth of new bacteriophages, from released ensilicated sample, within the bacterial host culture shows that the virulence of the ensilicated phages can be preserved.

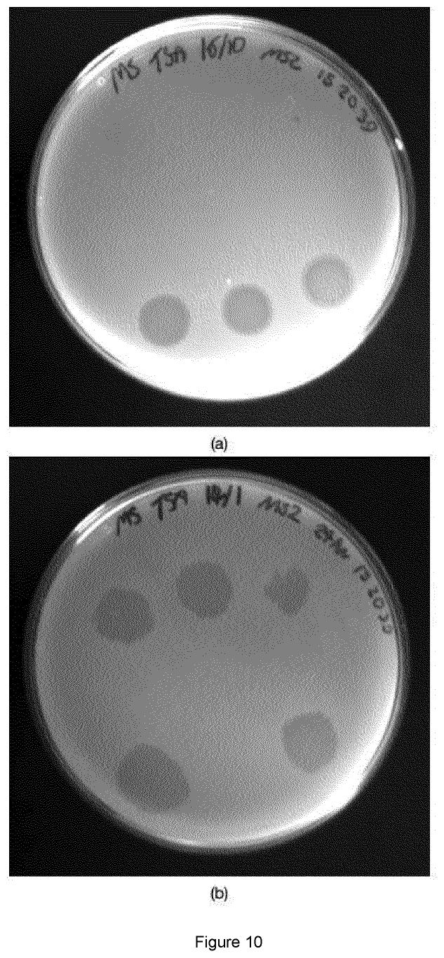

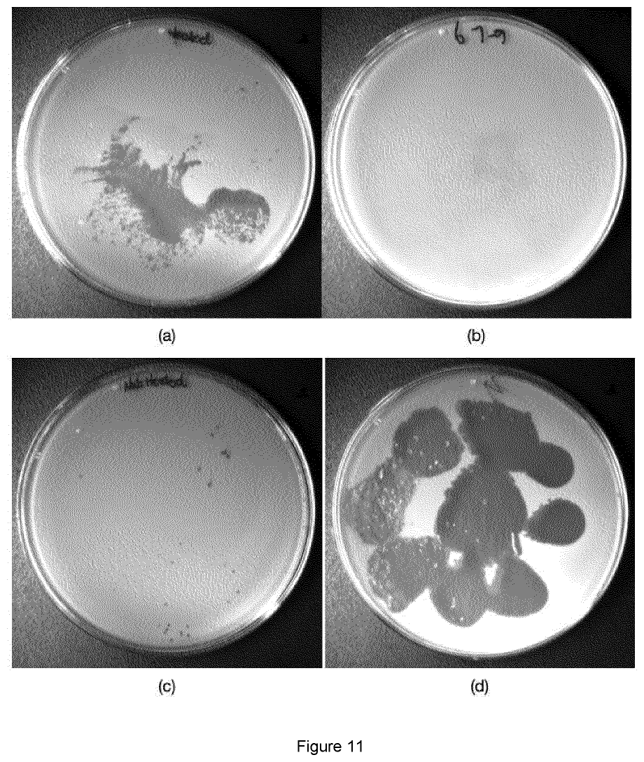

For ensilicated MS2 phages, lower levels of HF were also used to release the particles. HF concentrations of 1.5, and 3.0 mg/mL were used to release approximately 10 mg of ensilicated MS2 for one hour. 4 mL of each release was then added to 40 mL of 1 hour old E coli and incubated. Sampling was carried out at 5 hours and 24 hours of incubation time. Each sample was then plaque assayed as standard. Samples taken all produced plaques of a relatively high concentration. Each spot showed either full confluence or semi confluence (see FIG. 10). While an exact value for the titre cannot be established from the images obtained it is clear to see that MS2 is significantly more easily released, so could be replicated to a much higher titre than ensilicated phage K.

Release without HF

The release of ensilicated MS2 shows one major difference when compared to other ensilicated targets. When added to a mature bacterial host culture in a liquid suspension the phage is able to replicate without the prior removal of the silica shell using hydrofluoric acid. A small mass (40 mg) of ensilicated MS2 powders were added to 1 mL of TSBss and incubated for 1 hour before being added to 400 mL of 1 hour old E. coli and incubated for a further 24 hours. These samples were then plaque assayed. The results show high titre values of ensilicated MS2 without the use of hydrofluoric acid to break the silica shell. These values are of the same order of magnitude as with native unensilicated native MS2.

The ensilicated powders used were over 8 weeks old and had been stored at room temperature in Eppendorf vials. While there was no native control, stored for that length of time at the same temperature used during this experiment, it can be confidently stated that native MS2 lysates would lose all viability after this length of time in at 2-8° C., let alone at approximately 20° C. Consequently, it is clear that the MS2 growth from the ensilicated powders could not be attributed to adsorbed phages onto the surface of the silica and must be due to fully ensilicated bacteriophages being released in the TSB and bacterial culture.

### Stability Testinq

Using a suitable release method, the protection that the silica coat offered to phages was established by looking at encapsulated phage K.

Thermal stability characterisation of native phage K showed that the phage was unable to survive at temperatures higher than 70° C. for 30 minutes. Consequently, by heating the ensilicated phage at 90° C. for 30 minutes and then releasing the ensilicated phage, any plaque produced from the release would be purely those ensilicated rather and adsorbed onto the surface of the silica particles. It would also show that ensilication would offer much improved thermal stability as opposed to native liquid lysates. A sample of ensilicated phage K was heated in an oven for 30 minutes at 90° C., once cooled the ensilicated sample was released following the most successful set of parameters, before being plaque assayed alongside a heated native sample, an unheated native sample, and an unheated ensilicated and released sample. The results of this are shown in the scanned images of agar plates shown in FIG. 9.

For MS2, the ensilicated phage was also stored at room temperature over a period of several weeks. These conditions would cause the native liquid lysates to remove all viability since supplied guidance from ATCC indicates that phage MS2 is in liquid suspension, a rapid decrease in titre is expected when kept between 2-8° C. in as soon as 2 days. As a result, deep freezing is required for MS2 storage. These results demonstrate show that ensilication of MS2 is able to protect MS2 against thermal degradation.

### Porosity

### SYBR Safe Dye and BCA Testing

The use of a fluorescent DNA dye was used to determine if the porosity of the silica shell for ensilicated phage K is large enough to allow small to medium-sized molecules to pass through the channels and to interact with the virion encased within. A high level of florescence detected, when the dye is added to ensilicated phages, will show that the dye is able to penetrate the silica shell and successfully bind with the DNA either inside the phage capsid, or to free DNA in solution. As SYBR safe dye is not specific however it could also result in false positives from the DNA of contaminants in the solution, such as left-over bacterial DNA from propagation. The testing of phage K powders by SYBR safe dye is summarised in Table 2 below

TABLE 2
Table showing the results of florescence measurement of native
and ensilicated phage K samples with SYBR safe DNA dye. Vortexed
phage K are native enriched lysates that have been vigorously
vortex mixed to encourage the ejection of phage DNA.

| | Average Florescence, | 2 × RSD, |
|---|---|---|
| Sample | (AU) | (%) |
| Blank | 1414 | 1.13 |
| Native Phage K | 21921 | 25.16 |
| Vortexed Native Phage K | 29262 | 18.62 |
| Ensilicated Phage K without | 258 | 39.10 |
| Dye | | |
| Ensilicated Phage K | 37445 | 35.53 |

The results show that when mixed with the SYBR safe dye the ensilicated phage shows a level of florescence comparable to native phage lysates 37,445 AU against 21,921 AU respectively. Despite the relatively high uncertainty values for the florescence readings there is a significant difference between the results of ensilicated phage mixed with SYBR safe and the results of ensilicated phage without dye added that show the silica powder itself contributes very little to the florescence signal. As such it can be stated with confidence that the silica shell is porous enough to allow for the SYBR safe molecule to penetrate and interact with the DNA contained within.

### BCA Assay

Similar to the use of SYBR safe dye, the copper ions and necessary potassium tartrate in the BCA mixture should be able to travel through the pore channels in the silica coat and interact with the protein peptide backbone and amino acid residues, producing the pale blue, and bright purple complexes characteristic of the reaction. The results from the BCA assay of ensilicated phage K powders is shown in Table 3. This information can be further reinforced by carrying out a similar test using BCA reagents in place of SYBR safe dye.

TABLE 3
Table showing the results of absorbance measurement of BCA
analysis of native and ensilicated phage K materials.

| | Protein Concentration, | 2 × RSD, |
|---|---|---|
| Sample | (mg/mL) | (%) |
| Buffer | 0.11 | 64.08 |
| Native Phage K | 1.70 | 0.01 |
| Ensilicated Phage K without | 0.46 | 1.79 |
| BCA | | |
| Ensilicated Phage K | 0.36 | 28.28 |
| Ensilicated and Released | 1.23 | 5.90 |
| Phage K | | |

The results obtained from the BOA analysis of ensilicated phage powders shows that while the absorbance value for the ensilicated samples is much lower than that of native phage or released phage (1.70 or 1.23 mg/mL) it is still significantly larger than that of the buffer value (0.11 mg/mL). The value of the powder without BCA reagents added provides a value of the absorbance caused by the powder itself. With this value being much lower than the ensilicated sample, it again shows that the silica shell is porous enough to allow the required reagents to traverse the pore structure and produce the chelated copper complexes required for analysis. This data reinforces that collected from the SYBR safe dye analysis, proving the silica shell is porous enough to allow small/medium-sized molecules through to the virion.

### Nitrogen BET

Three ensilicated phage samples were also tested, each ensilicated using a different prehydrolysed silica: lysate ratio; 1:1, 1:10, and 1:40. Using the BET isotherm the surface area of the material can be calculated, these values can be compared between samples to show how porous they are. Further analysis using a Barrett-Joyner-Halenda (BJH) isotherm allows for a map of the pore size distribution, giving values for the number of pores of a particular size. The BET values for the samples are shown in Table 4.

TABLE 4
Table showing the surface area of native
and ensilicated phage K materials

| Sample | Surface Area, (m²/g) |
|---|---|
| Phage K 1:1 | 30.599 |
| Phage K 1:10 | 31.545 |
| Phage K 1:40 | 34.467 |

The surface areas of the samples show several key results; the surface area for all of the phage samples is larger than that for previously encapsulated materials, such as the lysozyme.

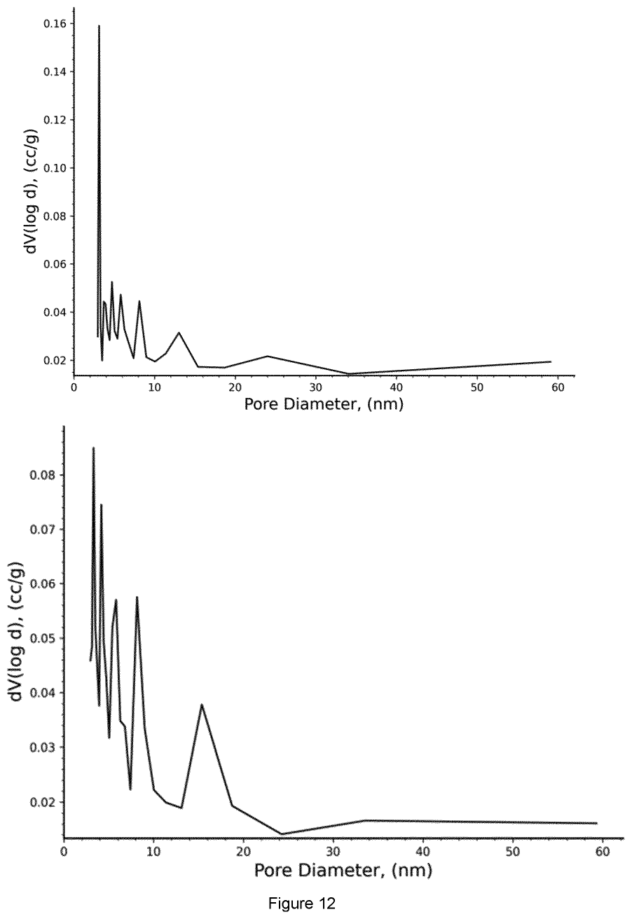

The BJH isotherms of the 1:10 and 1:40 sample is shown in FIG. 12. These graphs show that for ensilicated phage samples the vast majority of the pores in the samples are below 10 nm in diameter. Between the 0-10 nm range the pores are distributed in very tight clusters, producing sharp peaks showing a very small range in the diameter of these pore clusters.

Between 10-20 nm there is only one peak which is much broader than others and shows a more even distribution of pore sizes. Above 20 nm there is very little pore volume in the phage samples.

### Visualization Study of Silica Encapsulation by TEM

The large size of phage K permits the use of electron microscopy to view the silica growth over the length of the silica encapsulation or ensilication experiment. The ensilication method used for the visualisation experiments differs slightly from the ensilication method discussed above because the above-mentioned process produces micro particles very quickly and would be unsuitable for sampling to show differences in particle growth.

The differences used were aimed at decreasing the rate of the ensilication process. Particularly the pH of the reaction was reduced from 7.5 down to 6.00. As well as the reduction of pH the reaction was carried out in an ice bath so as to keep the temperature down to a level that would further reduce the reaction rate as well as providing an environment that promoted phage survivability. We elected for the time points used for the sampling of the modified ensilication process to not match the times of a regular process to make visualisation easier.

Samples were taken from a modified ensilication batch process at various time points, blotted and stained on carbon grids so as to halt the reaction to provide a freeze frame of the sample at that particular ensilication time point.

The images obtained from viewing each of the samples at time points across the ensilication process clearly showed the initial formation and growth of the silica coat on the phage virions and the incorporation of the virions into the silica networks characteristic of sol-gel processes. Furthermore, these images have a measurable size difference around key structural components of the phage molecules. This gives visual evidence on the initial growth sites of the silica on the molecule and the areas the silica favours. This gives a strong indication for our initial hypothesis that silica is attracted to the positively charged areas on the surface of the target during ensilication, and after that initial step, it grows and spreads all along the surface of the phage.

Each of the images collected from the TEM analysis of the ensilication samples were analysed using ImageJ software. Key points of the phage virions were measured allowing comparison of the physical sizes of the virions throughout the ensilication process. These key points were head diameter bottom to top, head diameter left to right, tail width averaged across the length of the tail from at least 5 measurements, and where a virion presented a straight tail, tail length from bottom of head to top of base plate.

### Key Characteristic Measurements

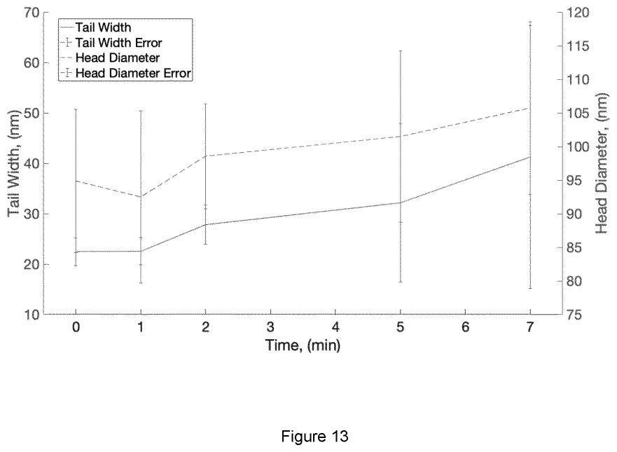

The measurements of the tail widths and head diameters were collected together and averaged for each time point, with the number of measurements based of the number of clearly visible virions at each time point. Tail width consisted of 15 measurements for 1 and 2 minutes, 25 measurements for native and 5 minutes, and the 7-minute time point had 10 measurements. Head Diameter measurements were 18 for 1 and 2 minutes, 30 for 5 minutes and native, and 12 measurements for 7 minutes. The error for the size measurements given is ±2 standard deviations of each time point, quoted as a percentage of the mean value. This data is shown in table 2 and depicted graphically in FIG. 13. The standard deviation for the tail width increases significantly as the silica growth increases. This is expected as the silica growth across the tail length forms specific localised nodule growths as well as the regular deposition of silica across the whole length of the tail, as such the measured width varies depending on where the measurements are taken.

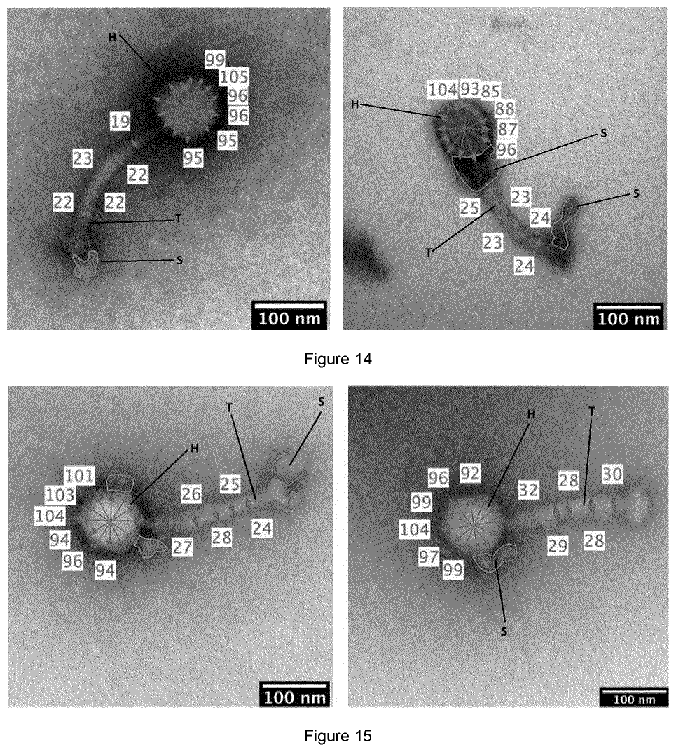

The images captured at each time point show a clear difference in the morphology and appearance of silica around the virions in the first stages of ensilication. The following FIGS. 14-17 show TEM images of bacteriophage K through various stages of ensilication. The key characteristic measurements are shown in red and annotated with relevant sizes in nm. The silica nodule growths are highlighted in yellow on each of the images. Even at samples taken at 1 minute of ensilication differences from the native samples are apparent. FIG. 14 shows some of the virions after 1 minute of ensilication. The superstructure of the phage is still largely visible, however the silica in solution begins to blur the fine details of the images. The beginnings of some silica growth are discernible however it was not yet significant enough to affect the physical measurements. The beginnings of nodule growth, particularly around the baseplates of the virions is more apparent in these images.

FIG. 15 shows TEM images collected after 2 minutes of ensilication. The initial silica growths faintly visible along the length of the tail and over the head of the phage in FIG. 10 become much larger and easily discernible at this time point. In addition to the increase of nodule growth size, the size of the overall silica coat at 2 minutes is large enough for it to be noticeable within the measurements of the phage structure, table 2. At this time there is an average difference of 18.94% within the width of the tail coat and 6.94% in the diameter of the head coat, between virions measured at 1 minutes and 2 minutes.

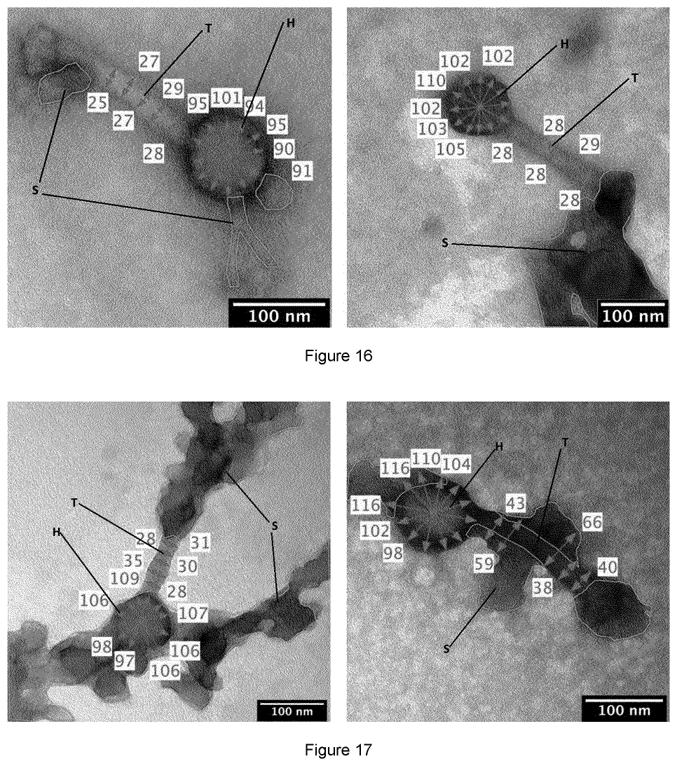

These measurements again increase in the images taken after 5 minutes of ensilication, although at a smaller rate, the virion average sizes see increase of 13.56% and 2.90% to the tail coat width and head coat diameter respectively. Both of these increases are smaller than the measurement error however (see FIG. 16). Virions observable in images captured after 7 minutes show a growth increase between that of 1-2 minutes and 2-5 minutes, with increases of 22.07% and 4.25% to the tail coat width and head coat diameter respectively (see FIG. 17). Due to issues with imaging the increasingly thick silica gel forming in the solution measurements after 7 minutes were not recorded.

### Silica Growth Preference

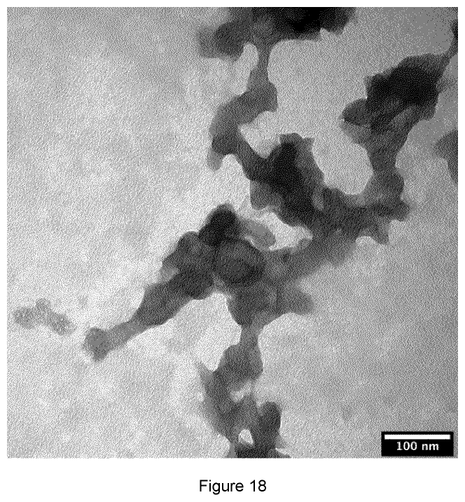

The baseplates of the phage molecules appear to be preferentially favoured by the silica monomers over the rest of the phage. The baseplate assemblies show a large mass of silica deposited after only 2 minutes. These masses of silica formed over the phage baseplate seem to be the beginnings of silica gel networks characteristic of the sol gel process used in ensilication. These networks incorporate the phage virions at the head and baseplate, before enveloping the whole virion. Comparing the data shown in table 5.1 alongside the captured images it is easy to see that the silica coat shows growth over the course of the experiment, not only in a uniform layer around the phage virion but in the irregular nodule growth, particularly around the baseplate and head group of the phages. The images collected show that ensilication is an extremely mild process involving a uniform deposition of silica cross the entire of a phage body and major non-uniform build-up of silica from several sites on the surface of the phage body and head. This non-uniform growth extends away from the initial nucleation site on the phage body and spans out to form silica networks characteristic of a modified sol-gel process. Each silica sphere could contain one or several ensilicated phages. This is reinforced by what is shown in FIG. 18, which shows an ITEM image of phage K after 7 minutes of ensilication. The outline shows 2 distinct individual phage virions being incorporated into the same silica cluster network.

The end result of the ensilication process under the above-mentioned standard ensilication, after 10 minutes at a standard rate of ensilication, is a collection of smooth networked spheroids or spheres approximately 400-500 nm in diameter, an electron micrograph of this is shown in FIG. 5. This figure was collected after the application of standard phage ensilication, as opposed to the modified phage ensilication used during the visualisation process.

The results of the porosity testing of ensilicated phage powders show that ensilicated bacteriophages are more porous than some previous biopharmaceutical targets, such as lysozymes.

### CONCLUSIONS

It was found that the ensilicated bacteriophage are able to produce plaques, even when stored at ambient temperatures, for periods of time much longer than native phage lysates. This shows that ensilicated bacteriophages are suitable for long term ambient storage, where native liquid phage lysates would otherwise degrade. To reinforce this, ensilicated phage powders are extremely resilient to high temperatures, losing no additional infectivity when compared to non-heated ensilicated powders. The TEM images collected from the visualisation of then ensilication of phage K show that the process is extremely mild and even a structure as complex as a tailed phage, comprising genetic material, is able to survive intact. The deposited silica seems to have no or minimal effect on the structural integrity of the virus.

As far as the present inventors are aware, this is the first time a similar silica encapsulation process has been applied to a biological molecule as large and complex as a full virion without the presence of an intermediate polycationic structure. Furthermore, the resultant particles were found to have different properties to previously ensilicated proteins or polypeptides. The particles had a much larger median size, while also being more porous.

The results obtained show ensilication is suitable for application to a wide range of viruses, including both i) bacteriophage K which is a DNA bacteriophage virus with a highly complex structure, and ii) MS2 which is an RNA bacteriophage virus with a less complex structure. MS2 has large structural similarity to many mammalian viruses, thus this work also serves as a proof-of-concept that these methods can be used for mammalian viruses, particularly other simple and spherical viruses such as influenza and rotavirus.

Notably, the present inventors found that enrichment of the virus lysate was required before ensilication. While an enrichment method using PEG 6000 or chloroform are described above, the present inventors consider that a different purification or enrichment method could be used prior to ensilication. The present inventors also found that certain parameters of the enrichment and/or ensilication method could result in the highest surviving release titres.

Even when subjected to ensilication and release the infectivity of phage K can be considered infectious and preserved. Fresh phage stocks can be grown indefinitely from the released phage solution. When applied to virulent subjects, ensilication has no permanent detriment to the infectivity of the virus, as the virus is able to replicate and replenish its virion level. Furthermore, the bacteriophage was ensilicated and stored for long periods of time at room temperature while retaining its infectivity. Across the study phage K showed a loss of approximately 4 logs in concentration. Considering how unstable the phages are at room temperature, both the ensilicated phages showed much greater resilience to extreme temperatures. No additional loss of titre after being heated at 90° C. for 30 minutes was observed. This indicates that the survival of phages at toom temperature is improved as compared to native lysates, indicating that the ensilicated samples can remain viable for use for extended periods of time without refrigeration or cold-chain.

### Supplementary Information

### Detailed Materials and Characterisation Methods

Tryptic Soy Agar (TSA) from Sigma-Aldrich contains Agar 15 g/L, casein peptone (pancreatic) 15 g/L, sodium chloride 5 g/L, soya peptone (papainic) 5 g/L. Made up in ddH₂O, autoclaved at 121° C., 15 minutes Tryptic Soy Broth (TSB) from Sigma-Aldrich contains casein peptone (pancreatic) 17 g/L, dipotassium hydrogen phosphate 2.5 g/L, glucose 2.5 g/L, sodium chloride 5 g/L, soya peptone (papain digest) 3 g/L. Made up in ddH₂O, autoclaved at 121° C., 15 minutes.

Tryptic Soy Broth with added salts (TSBss) from Sigma-Aldrich contains casein peptone (pancreatic) 17 g/L, dipotassium hydrogen phosphate 2.5 g/L, glucose 2.5 g/L, sodium chloride 5 g/L, soya peptone (papain digest) g/L. NaCl 5.7 g/L, MgSO₄·7H2O 2.09 g/L added additionally. Made up in ddH₂O, autoclaved at 121° C., 15 minutes.

PEG-6000 purchased from VWR. NaCl, MgSO₄·7H₂O, gelatine, and glycine purchased from Fisher Scientific. Bacteriophage K and host Staphylococcus aureus H560 were provided by Dr. S. Milo and Prof. A. Jenkins, University of Bath.

Saline magnesium buffer (SM buffer) contains NaCl 5.7 g/L, MgSO₄·7H₂O 2.09 g/L, Trizma base 7.88 g/L, gelatine 0.1 g/L. pH 7.5 with HCl, made up with ddH₂O, autoclaved at 121° C. for 15 minutes

Saline magnesium buffer with glycine (SM(G) buffer) contains NaCl 5.7 g/L, MgSO₄·7H₂O 2.09 g/L, Glycine 3.75 g/L, gelatine 0.1 g/L. pH 7.5 with HCl, made up with ddH₂O, autoclaved at 121° C. for 15 minutes.

Tris buffer contains Trizma base 7.88 g/L, made up with ddH₂, autoclaved at 121° C. for 15 minutes.

Bacteriophage MS2 lyophilised lysates—Obtained from ATCC, product no. 15597-B1, batch no. 70008569

### Other Materials

UA Zero Stain—Agar Scientific
200 Mesh, Copper Supported Carbon/Formva Electron Microscopy Grids—Agar Scientific
Sodium chloride—Fisher Scientific UK
Magnesium sulphate heptahydrate—Fisher Scientific UK
Trizma base—Sigma Aldrich
Gelatine Type B From Bovine Skin—Sigma Aldrich
Hydrochloric Acid (32%)—Sigma Aldrich
Polyethylene glycol M/W 6000 (PEG-6000)—Sigma Aldrich
Tetraethyl orthosilicate—Sigma Aldrich
Sodium Fluoride—Sigma Aldrich
Glycine—Fisher Scientific UK

### Visualisation of Ensilication by TEM

The saline magnesium (SM) buffer was adjusted to pH 6. A 1:100 ratio of prehydrolysed silica to this SM buffer was used. 1000 μL of enriched 0.22 μm filtered phage K lysate was transferred to a 1.5 mL Eppendorf vial. To this 100 μL of prehydrolysed silica was added and a timer started. The Eppendorf vials were gently inverted. After 45 seconds sample was taken and immediately blotted onto freshly glow discharged carbon grids. 1-2 drops of 1% uranyl acetate stain was used. The total sampling process took approximately 15 seconds, meaning that each sample represented 60 seconds of ensilication time. Multiple samples were taken from the same ensilication run and analysed using a Tecnai 12 TEM fitted with a BioTwin Spirit objective lens system and FEI Eagle 4k×4k CCD Camera at 120 kV acceleration voltage (University of Bristol, Wolfson Bio-imaging Facility). Where required to obtain a wider range of images some sample times were repeated.

### TEM

Using freshly glow-discharged carbon grids a small volume of lysate (5-10 μL) was pipetted onto the grid. The sample was allowed to settle onto the grid for 30 seconds before excess liquid was wicked away using filter paper. The grid was washed twice with ddH2O, with the excess water being wicked away as before. 2 drops of 1% uranyl acetate solution or UAZero Stain (Agar Scientific) were added to the grid and left to set for 30 seconds before the excess was carefully wicked away with filter paper. The grids were then left to air dry for approximately 3 hours before use.

The TEM used was JEOL/EO JEM-2100+ fitted with a LaB₆ Electron gun and an acceleration voltage of 120 kV used (University of Bath, Materials Analysis Suite). Grids used were 200 mesh formva/carbon copper supported grids from Agar Scientific. TEM used was Tecnai 12 TEM fitted with a BioTwin Spirit objective lens system and FEI Eagle 4k×4k CCD Camera at 120 kV acceleration voltage (University of Bristol, Wolfson Bio-imaging Facility. Grids used were 200 mesh formva/carbon copper supported grids from Agar Scientific.

### FE-SEM

Fresh carbon/mica film was stuck to SEM sample holders. Dried ensilicated powder was milled to appropriate size using a mortar and pestle, before being spread onto the film. The samples and holders were stored under vacuum until use. Immediately before being analysed each sample was sputter coated with 20 μm layer of chromium. Sputter coated used was Quorum 150VS Plus, SEM instrument used was JEOL FESEM6301 F (University of Bath, Materials Analysis Suite) with a field emission electron gun and using acceleration voltage of 5 kV

### Dynamic Light Scattering

All samples used were filtered through 0.22 μm Millex syringe filters into disposable polystyrene micro cuvettes that had been thoroughly rinsed with both ddH2O and the samples. The samples were analysed using a variety of sampling times and repeats, each time at standard temperature and pressure, using a 173° measurement angle. The instrument used was a Malvern Panalytical Zetasizer Nano ZS fitted with a 633 nm He-Ne laser DLS analysis was used to determine the monodispersity of the lysate. The Malvern Panalyticals Zetasizer software uses a metric called ‘polydispersity index’ (PDI) to indicate the purity of samples, values closer to 0 are purer while values closer to 1 are impure and a mixture of many different particle types

### Bicinchoninic Acid (BCA) Assay

Using a 96 well micro titre plate. 100 μL of 0.2 M NaOH was added to 100 μL of 2 mg/mL bovine serum albumin (BSA) and mixed thoroughly. This was then diluted to produce solutions of 0.8, 0.6, 0.4, 0.2, and 0 mg/mL of BCA in 0.1 M NaOH. 10 μL of these dilutions were then pipetted into the micro-titre plate to produce a calibration series ranging from 1.0-0 mg/mL BSA. The protein solution to be analysed was pipetted neat or diluted with 0.1 M NaOH to bring the concentration in range of the BSA calibration series, depending on the estimated concentration of the protein solution.

The BCA solution was prepared by mixing BCA reagent A and BCA reagent B in a 50:1 ratio and subsequently pipetting 200 μL of this mixture into each of the wells on the 96 well plate. The plate was then covered and incubated at 37° C. for 30 minutes before being analysed on a micro plate reader (PHERAstar FS, BMG LABTECH)

### DNA Fluorescence

DNA dye used was Invitrogen—SYBR Safe DNA Gel Stain. 10,000×Concentrate in DMSO. Batch No.: 1988947 Samples were pH adjusted using either HCl or NaOH to reach pH between 7-8. 900 μL of sample was pipetted into Eppendorf vials. SYBR safe dye was diluted by twice, using 10 μL of dye in 990 μL of Milli-Q water, and subsequent 100 μL of this dilution in 900 μL of Milli-Q water, to reach a final dilution of 1,000 times. 100 μL of this was added to the Eppendorf vials with the samples. Vials were gently inverted several times before having 200 μL pipetted into black plastic 96 well plates. These 96 well plates were incubated at 37° C. for 5 minutes before being analysed using microplate reader (PHERAstar FS, BMG LABTECH) at 509 nm.

### Sodium Dodecyl Sulphate (SDS) Pulsed gel Electrophoresis (PAGE)

Phage samples were run using a pre-cast gradient gel (Bio-Rad Mini-PROTEAN TGX Precast 8-16%) along with Novex Sharp unstained protein standard (Life Technologies, Thermo Fisher Scientific) as a reference ladder in the left most and right most wells. In the event of the gel warping these ladders can be compared and results extrapolated. 20 μL of phage lysate samples were added to 10 μL of SDS-PAGE sample buffer before being incubated at 95° C. for 10 minutes. The precast gel was secured into an assembly cassette (Mini Protean 3, Biorad) and electrophoresis buffer was added after which the reference ladders and phage samples were pipetted into the sample lanes. Cassette was run at below 200 V for 45 minutes. Once completed the gel was removed from the cassette and stained with approximately 20 mL of PageBlue at room temperature with rocking, with subsequent destaining conducted with ddH₂O at room temperature with rocking. The gel was imaged with Epi Chemi II Darkroom (UVP), fitted with Hamamatsu C4742-98 digital camera using ClearLive software.

### Differential Scanning Calorimetry (DSC)

A 700 μL sample of enriched, native, phage (e.g., phage K) was pipetted into stainless steel cells and placed into the instrument alongside a control containing blank SM buffer. The samples were heated on a ramp by 1° C. from 20° C. to 150° C. before being cooled back to 20° C. again.

### Nitrogen Physisorption, BET and BJH

All samples were measured careful into glass sample tubes and thoroughly degassed overnight Apparatus was leak tested prior to each sample analysis. Gas type used was N₂ at 77 K. Tolerances and equilibration times are shown in table 5. Instrument used was Quantachrome Anton Paar—Autosorb—iQ-C

TABLE 5
Tolerances and equilibration times used for the nitrogen
physisorption analysis of ensilicated bacteriophages.

| Measurement | | Equilibration Time, |
|---|---|---|
| Decade | Tolerance | (mins) |
| 10-7 & 10-6 p/p0 | 5 | 8 |
| 10-5 & 10-4 p/p0 | 3 | 6 |
| 10-3 p/p0 | 1 | 4 |
| Above 10-2 p/p0 | 0 | 2 |

### High Temperature Stability Tests

1 mL of phage lysates and 50 mg of ensilicated phages were placed in small vials. These were placed in an oven at 90° C. and heated for 30 minutes. The ensilicated sample was released as indicated above and both native and ensilicated released samples were plaque assayed.

### Long Term Stability Tests

10 mL of phage lysates and 50 mg of ensilicated phages were placed in small vials. These vials were left in ambient laboratory atmosphere. At intermittent dates 100 μL of native lysates were sampled, diluted with SM buffer and plaque assayed to determine loss of phage over time at room temperature. At the point where no PFUs from the phage were detected the ensilicated samples were released and plaque assayed to determine their titre.

### pH Characterisation

100 mL of pH adjusted SM buffer was used per pH point, the pH was adjusted using either HCl diluted in milli-q water, or using NaOH dissolved into milli-q water. To each of the pH adjusted buffers 1 mL of enriched phage lysates were added and the mixtures were stirrer at 125 RPM for 30 minutes. Immediately after stirring the mixtures were diluted down to a standard plaque assay range (up to ×10⁻⁹) in pH 7.5 SM buffer and a standard plaque assay carried out.

What is claimed is:

1. A particle comprising a virus encapsulated in an amorphous silica shell, wherein the amorphous silica shell is directly deposited about the surface of the virus.

2. The particle of claim 1, wherein the particle has a median diameter of at least 250 nm as determined by field-emission scanning electron microscope, optionally wherein the particle has a median diameter from 250 nm to 1000 nm, or preferably from 300 nm to 500 nm.

3. The particle of claim 1, wherein the virus is capable of replication in a host cell.

4. The particle of claim 1, wherein the particle is free of polylysine

5. The particle of claim 1, wherein the silica shell has a spheroid morphology.

6. The particle of claim 1, wherein the virus encapsulated in the silica shell is protected from surrounding conditions, optionally wherein the surrounding conditions are temperature or pH.

7. The particle of claim 1, wherein the virus encapsulated in the silica shell is thermally stable, preferably wherein the virus is stable after heating at 90° C. for 30 minutes.

8. The particle of claim 1, wherein the virus is a DNA virus or an RNA virus.

9. The particle of claim 1, wherein the virus is a bacteriophage.

10. The particle of claim 9, wherein the virus is (i) a myoviridae virus, for example, bacteriophage K or (ii) wherein the virus is a fiersviridae virus, for example, phage MS2.

11. The particle of claim 1, wherein the virus is a mammalian virus.

12. A method of producing a virus encapsulated in an amorphous silica shell, the method comprising
enriching or purifying a virus, and suspending the virus in buffer, hydrolysing a silica precursor
directly contacting the hydrolysed silica precursor with the surface of the virus in buffer and encapsulating the virus in an amorphous silica shell

13. The method of producing the particle of claim 12, wherein the virus in the buffer is present at a concentration greater than 1×10⁷ PFU/ml, more preferably from about 1×10⁸ PFU/ml to about 1×10¹⁰.

14. The method of producing a virus of claim 12, wherein the buffer comprises one or more salts containing one or more of a magnesium salt, a calcium salt, or a sodium salt, optionally wherein the buffer is an SM buffer, a modified SM buffer, PBS, an imidazole buffer, a sucrose buffer, or bis-tris buffer.

15. The method of producing the particle of claim 12, wherein the enriching or purifying is by PEG-based enrichment.

16. The method of producing the particle of claim 12, wherein the virus in buffer has a polydispersity index (PDI) of less than 0.2, more preferably less than 0.15

17. The method of producing the particle of claim 12, wherein the silica precursor is a tetra-alkyl orthosilicate, preferably tetra-ethyl orthosilicate (TEOS).

18. The method of producing the particle of claim 12, wherein the ratio of hydrolysed silica to the virus in buffer is from about 1:75 to 1:150, for example, about 1:100

19. The method of producing the particle of claim 12, wherein the contacting is carried out for at least 5 minutes, preferably at least 10 minutes and/or wherein the contacting is carried out at a pH greater than 7, preferably between 7.25 and 8.5, for example, about 7.5

20. Use of the method of claim 12, wherein the method is used for preservation or storage of a virus.

21. A particle formed by the method of claim 12.

22. A dry powder comprising the particles of claim 1.

23. A pharmaceutical composition comprising (i) one or more particles of claim 1 and/or a dry powder comprising the particle of claim 1, or (ii) at least two particles of claim 1, wherein the at least two particles comprise different viruses.

24. The particle of claim 1, the dry powder comprising the particle of claim 1, or a pharmaceutical composition comprising (i) one or more particles of claim 1 and/or the dry powder comprising the particle of claim 1, or (ii) at least two particles of claim 1, wherein the at least two particles comprise different viruses, for use in therapy, preferably for use as an antibacterial medicament wherein the virus is a bacteriophage.

25. Non-therapeutic use of the particle of claim 1 a dry powder comprising the particle of claim 1, or a pharmaceutical composition comprising (i) one or more particles of claim 1 and/or the dry powder comprising the particle of claim 1, or (ii) at least two particles of claim 1, wherein the at least two particles comprise different viruses, for use as an antimicrobial, wherein the virus is a bacteriophage.
